(19) United States
(12) Patent Application Publication
(10) Pub. No.: US 2022/0025015 A1
(43) Pub. Date: Jan. 27, 2022

(54) Methods, compositions and therapeutical vaccine for autoimmune diseases and allergy treatment

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 17/495,639

(22) Filed: Oct. 6, 2021

Related U.S. Application Data
(63) Continuation-in-part of application No. 16/380,951, filed on Apr. 10, 2019.
Application No. 16/380,951 is a continuation-in-part of application No. 15/723,173, filed on Oct. 3, 2017, now abandoned.
Application No. 15/723,173 is a continuation-in-part of application No. 17/344,932, filed on Jun. 10, 2021.
Application No. 17/344,932 is a continuation-in-part of application No. 16/029,594, filed on Jul. 7, 2018, now abandoned.
Application No. 16/029,594 is a continuation of application No. 16/566,716, filed on Sep. 10, 2019, now abandoned.
Application No. 16/566,716 is a continuation-in-part of application No. 17/385,908, filed on Jul. 27, 2021.
Application No. 17/385,908 is a continuation of application No. 16/819,168, filed on Mar. 16, 2020, now abandoned.
(60) Provisional application No. 63/111,018, filed on Nov. 7, 2020.

(51) Int. Cl. C07K 14/74; A61P 37/00

(52) U.S. Cl. CPC C07K 14/70539; A61K 38/00; A61P 37/00

(57) ABSTRACT

Compositions, reagents, formulations and methods to treat disease including autoimmune diseases and allergy are described. The compositions comprise an antigen causing immune intolerance, an immunosuppressant in a sustained release formulation. The methods, compositions, formulations and reagents to treat allergy also relate to applying the combination of allergen and immune activity enhancing agent in a sustained release formulation to a subject in need.

### CROSS-REFERENCE TO RELATED APPLICATIONS

This application claims priority to U.S. Provisional Patent Applications 63/111,018 filed on Nov. 07, 2020, 63/121,974 filed on Dec. 06, 2020 and 63/130,396 filed on Dec. 23, 2020. It is also a Continuation-In-Part application of U.S. application Ser. No. 15/723,173, Ser. No. 16/380,951, Ser. No. 16/029,594, Ser. No. 17/344,932, Ser. No. 16/566,716, Ser. No. 16/819,168 and Ser. No. 17/385,908. The entire disclosure of the prior applications is considered to be part of the disclosure of the instant application and is hereby incorporated by reference.

### BACKGROUND

### Field of the Invention

The current invention relates to compositions, formulations and reagents to treat diseases including autoimmune disease and allergy. The current invention also discloses methods to treat autoimmune disease and allergy. The compositions to treat autoimmune disease and allergy relate to combination of disease related antigen and immunosuppressant in a sustained release formulation.

The methods, compositions, formulations and reagents to treat allergy also relate to applying the combination of allergen and immune activity enhancing agent in a sustained release formulation to a subject in need.

### Background Information

Immune responses are necessary for protection against potentially pathogenic microorganisms. However, undesired immune activation can cause injurious processes leading to damage or destruction of one's own tissues. Undesired immune activation occurs, for example, in autoimmune diseases where antibodies and/or T lymphocytes react with self-antigens to the detriment of the body's tissues. This is also the case in allergic reactions characterized by an exaggerated immune response to certain environmental matters and which may result in inflammatory responses leading to tissue destruction. This is also the case in rejection of transplanted organs which is significantly mediated by alloreactive T cells present in the host which recognize donor alloantigens or xenoantigens. Immune tolerance is the acquired lack of specific immune responsiveness to an antigen to which an immune response would normally occur. Typically, to induce tolerance, there must be an exposure to a tolerizing antigen, which results in the death or functional inactivation of certain lymphocytes. This process generally accounts for tolerance to self-antigens, or self-tolerance. Immunosuppressive agents are useful in prevention or reduction of undesired immune responses, e.g., in treating patients with autoimmune diseases or with allogeneic transplants. Conventional strategies for generating immunosuppression associated with an undesired immune response are based on broad-acting immunosuppressive drugs. Unfortunately, the use of broad-acting immunosuppressants is associated with a risk of severe side effects, such as tumors, infections, nephrotoxicity and metabolic disorders. Accordingly, new immunosuppressant therapies would be beneficial.

### DESCRIPTION OF THE INVENTIONS

Previous U.S. application Ser. No. 15/723,173, Ser. No. 16/380,951, Ser. No. 16/029,594, Ser. No. 16/566,716, Ser. No. 16/819,168, Ser. No. 17/344,932 and Ser. No. 17/385,908 by the current inventor disclose methods, agents, devices and compositions to treat autoimmune diseases and allergy, and to prevent antigen specific antibody generation including anti-drug antibody generation. The agents in the previous US applications include antigen-drug conjugate such as antigen- immunosuppressant molecule conjugate. The agents and compositions can be a mixture of antigen and immunosuppressant molecule or their conjugate. They can be in the form of linear polymer, microparticle, nanoparticle, liposome, implant or a transdermal drug delivery system such as a transdermal patch. Examples of the antigen include B cell antigen, T cell antigen in MHC-peptide complex form or the antigen peptide (or its derivative) that can bind with MHC. A carrier system can be used for the previous and current applications to construct the conjugate. For example, the liposome or microparticle or nanoparticle can be used as a carrier. The antigen can be immobilized on the surface of the liposome or particles and the effector molecule (e.g. alpha-gal, rhamnose, immune suppression cytokine, tregitope peptide, toxin, siRNA or miRNA or the like, immune suppressant, antisense molecule) can be either encapsulated inside or co-immobilized on the surface of liposome or particles. The carrier can also be a linear or branched polymer such as dextran, hyaluronic acid, heparin, chondroitin sulfate and polypeptide. Both antigen and the effector molecule (such as immunosuppressant) can be conjugated to the polymer. They can be given to the subject in need to treat autoimmune diseases and allergy or inhibit anti-drug antibody production or induce antigen specific immune tolerance by administering to the subject said conjugate (e.g. subcutaneous or intralymphatic injection or applied to the skin such as the skin of upper arm).

Additional details can be found in the previous disclosures.

In one aspect, the current invention discloses compositions and formulations comprising one or more antigen causing disease condition and one or more immunosuppressant in a sustained (extended) release system such as an in-situ gelling system or implant to treat said disease condition selected from autoimmune disease, allergy and anti-drug antibody. The current invention also discloses a method to treat autoimmune disease or allergy or inhibit anti-drug antibody production or induce antigen specific immune tolerance in a subject by administering to the subject said compositions and formulations as an injection.

In one aspect, the current invention discloses compositions and formulations comprising one or more antigen causing disease condition and a vaccine adjuvant type agent (e.g. TLR agonist,

STING agonist) in a sustained (extended) release system such as an in-situ gelling system or implant to treat said disease condition selected from autoimmune disease, allergy and anti-drug antibody. The current invention also discloses a method to treat autoimmune disease or allergy or inhibit anti-drug antibody production or induce antigen specific immune tolerance in a subject by administering to the subject said compositions and formulations as an injection.

In one aspect, the current invention discloses compositions and formulations comprising one or more antigen causing disease condition in a sustained (extended) release system such as an in-situ gelling system or implant to treat said disease condition selected from autoimmune disease, allergy and anti-drug antibody. Said compositions and formulations contains no vaccine adjuvant type agent and no immunosuppressant. The current invention also discloses a method to treat autoimmune disease or allergy or inhibit anti-drug antibody production or induce antigen specific immune tolerance in a subject by administering to the subject said compositions and formulations as an injection.

The composition and formulation of the current invention can be in a gel form or high viscosity liquid or solid form or implant form. Gels are used herein refer to a solid, jelly-like material that can have properties ranging from soft and weak to hard and tough. As is well known in the art, a gel is a non-fluid colloidal network or polymer network that is expanded throughout its whole volume by a fluid. A hydrogel is a type of gel which comprises a network of polymer chains that are hydrophilic, sometimes found as a colloidal gel in which water is the dispersion medium. Hydrogels are highly absorbent and can contain a high degree of water, such as, for example greater than 90% water. In some embodiments, the gel described herein comprises a natural or synthetic polymeric network. In some embodiments, the gel comprises a hydrophilic polymer matrix. In other embodiments, the gel comprises a hydrophobic polymer matrix. In some embodiments, the gel possesses a degree of flexibility very similar to natural tissue. In certain embodiments, the gel is biocompatible and absorbable. In certain embodiments, the gel is formed after being administered to the patient.

The composition and formulation can contain viscosity enhancing agent to increase its viscosity, which acts as a sustained release formulation. In certain embodiments, the formulation is a viscous liquid. In certain embodiments, the injection formulation has a viscosity greater than 5,000 cps at room temperature. In certain embodiments, the injection has a viscosity greater than 50,000 cps at room temperature. In certain embodiments, the injection has a viscosity greater than 500,000 cps at room temperature. In certain embodiments, the injection has a viscosity of 5,000,000 cps at room temperature. Example of the viscosity enhancing agent can be found readily from known pharmaceutical acceptable excipients such as hyaluronic acid (linear or cross-linked form), HPMC, MC, CMC, starch and carbomer. In some embodiments, the viscosity enhancing agent is biodegradable.

In some preferred embodiments, the composition of the current invention is in a sustained release system to release the active drug within in an extended period of time, e.g. 50% drug (such as antigen, immunosuppressant, TLR agonist) released in several days to several weeks. The formulation is an extended (sustained) release formulation. In some preferred embodiments, the composition of the current invention is within a in situ gelling system and the formulation is said drug loaded in situ gelling formulation. In situ gelling systems are often polymeric formulations that are in solution (sol) forms before entering in the body, but change to gel forms under the physiological conditions. The sol-gel transition depends on one or a combination of different stimuli, like pH change, temperature modulation, solvent exchange, ultra violet irradiation and the presence of specific ions or molecules. Drug delivery systems having such properties can be widely used for sustained delivery vehicle preparation of the bioactive molecules. Some important advantages of these smart systems are ease of application and reduced frequency of administration, as well as protection of drug from environmental condition changes. Various natural and synthetic polymers undergo in situ gel forming and potentially can be used. Pectin, xyloglucan, gellan gum, chitosan and alginic acid are some of the natural polymers. The pectin gelation occurs in the presence of calcium ions. Xyloglucan exhibits thermally reversible gelation with body temperature. Dilute aqueous solutions of alginates form firm gels, on addition of di and trivalent metal ions, such as the Ca²⁺ in body fluid. Examples of alginate can be used include sodium alginate, potassium alginate, ammonium alginate and other pharmaceutically acceptable amine salt of alginate. For example, sodium alginate and hydroxypropyl methyl cellulose can be used in the in situ gel formulation. In situ gel formation of gellan gum occurs due to temperature modulations or the cations induced. Temperature and ionic condition (Ca²⁺) in body fluid cause a transition of aqueous solution of gellan into the gel state. Carbopol (poly acrylic acid) is a well-known pH dependent polymer, which stays in solution form at acidic pH but forms a low viscosity gel at alkaline pH. An in-situ gel can be formulated using carbopol and hydroxypropyl methylcellulose (HPMC). The latter is used to impart the viscosity to the carbopol solution, while reducing its acidity. Aqueous solution of carbopol-HPMC system is also an in situ gelling system. Pluronic F-127 is a triblock copolymer with nonionic nature, which undergoes in situ gelation by temperature change. It can be used together with Carbopol 934 and HPMC to prepare in situ gel. Chitosan aqueous solution forms a hydrated gel, like precipitate, at pH exceeding 6.2. Adding polyol salts, bearing a single anionic head, like glucose phosphate salts to chitosan aqueous solution can transform the cationic polysaccharides solution into thermally sensitive pH dependent gel. The sol form of such formulation (at the room temperature) turns into gel implants, when injected in vivo. Examples of them can be found in PMID: 25237648 and can be readily adopted for the current invention. In some embodiments, the gel is made of hyaluronic gel with optional calcium salt or ferric salt, for example the calcium ions and hyaluronic gel material is characterized in that: comprise hyaluronic acid, CaCl₂ or FeCl₃, and deionized water at weight ratio 0.01˜10:0.01˜10:100.

Examples of the in-situ gelling polymers used in in situ gelling system include chitosan, alginic acid, xyloglucan, gellan gum, sodium hyaluronate, pectin, hydroxypropyl methylcellulose (HPMC), methylcellulose (MC), carboxymethylcellulose, cellulose acetate phthalate (CAP), PGA, prolifeprospan, Carbopol, Pluronics, poly(lactide-co-glycolide) (PLGA), poly(D,L-lactide-co-hydroxymethyl glycolide) (PLHMGA).

The drug loaded in situ gelling system can use pH triggered in situ gelling polymers: pH triggered in situ gelling systems are solutions, which upon exposure to the pH of the body fluid converts into the gel phase e.g. such as carboxymethylcellulose, hyaluronate, cellulose acetate phthalate and Carbopol. Cellulose acetate phthalate latex remains free flowing solution at acidic pH (˜pH 4) and transform into the gel at neutral pH (pH7). Polyacrylic acid commercially known as Carbopol is a widely used polymer undergoes sol to gel transition in aqueous solution as the pH is raised above its pKa of about 5.5. the formulation of these type of system can have a low pH (4-5) to remain solution and become gel once inside body due to the pH change. Polyacrylic acid (e.g. Carbopol® 934) can be used as the gelling agent with HPMC (Methocel K4M) as viscosity enhancer. Polyacrylic acid (Carbopol) can be used as the gelling agent in combination with chitosan (as viscosity enhancer). The 0.4% w/v Carbopol/0.5% w/v chitosan based in situ gelling system is in liquid state at room temperature and at the pH of formulation i.e. pH 6.0, and underwent rapid transition into the viscous gel phase at pH 7.4 inside body.

The drug loaded in situ gelling system can use temperature triggered in situ gelling polymers: temperature triggered in situ gelling polymers remains liquid at low temperature (below 20° C.) and undergoes gelation at physiological temperature (35-37° C.). Following are some examples of temperature triggered in situ gelling polymeric systems: Poloxamers: Poloxamers, commercially known as Pluronic®, are the thermoreversible polymers commonly used for formation of thermosensitive in situ gelling systems. Upon heating from 4° C. to 23° C. or more, aqueous solution of Pluronic F127 or Poloxamer 407 at a concentration of 15%, transformed to a semisolid gel from a low viscosity solution. For example, the system can contain 20% w/w Poloxamer 407 and 10% w/w Poloxamer P188. A low viscosity aqueous solution of Poloxamer 407 (P407), at a concentration of 18% w/w (a 7:3 ratio of PEO and PPO) can be converted to a gel under the ambient temperature and the addition of hyaluronic acid (HA) in the Poloxamers blends can delay the gelation temperature by few degree Celsius and at specific concentration of Poloxamer/HA it is possible to get a thermoreversible gel with a gelation temperature close to body temperature. Viscosity enhancing agents (HPMC, MC and CMCNa) can be added to the 15% w/w PF-127 to form hydrogel, for example 15% PF-127 formulations containing 3% methylcellulose can be used as a temperature triggered in situ gelling system to load drug.

Poloxamines is another temperature triggered in situ gelling system, commonly known as Tetronics (tetra functional block copolymers of ethylene and propylene oxide), e.g. tetronic-oligolactide copolymer (made of Tetronic®1307 and pure L-lactide).

Another temperature triggered in situ gelling system is cellulose derivatives: ethyl (hydroxyl ethyl) cellulose, methylcellulose and HPMC are some of the cellulose derivatives which are being used as in situ gelling polymers. Aqueous solutions of ethyl (hydroxyethyl) cellulose (EHEC) exhibit thermosensitive gelation. On addition of sodium dodecyl sulphate or cetyl triammonium bromide, EHEC (1%-4% w/w) solutions undergoes sol-to-gel phase transition upon heating to 30-40° C. and forms stiff and clear gels. Some cellulose derivatives remain liquid at low temperature and become gel upon heating, for example aqueous solutions of methylcellulose and HPMC undergoes phase transition into gels between 40-50° C. and 75-90° C. respectively. However, phase transition temperatures of methylcellulose and HPMC are higher than the physiological temperatures, but can be lowered by making chemical or physical changes in the polymers. For example, addition of NaCl in methylcellulose or lowering the hydroxypropyl molar substitution of HPMC, the phase transition temperatures can be reduced to 32-34° C. and 40° C., respectively in these polymers.

The gelation temperature of 1% methylcellulose solution is decreased to the physiological temperature i.e. 37° C. by addition of fructose and sodium citrate tribasic dihydrate (SC) in different proportions. 1 to 5% SC can be added in the methylcellulose (1%) and fructose (10%) as the temperature triggered in situ gelling system.

Xyloglucan, a polysaccharide obtained from tamarind seed and approved for use as food additive. Partially degraded xyloglucan by β-galactosidase to >35% galactose removal ratio exhibits thermally reversible gelation in dilute aqueous solutions. The sol-gel transition temperature of xyloglucan varies with degree of galactose elimination and polymer concentration and related inversely, for example, on increasing the galactose removal ratio from 35 to 58% the sol-gel transition of xyloglucan was observed to be decreased from 40° C. to 5° C. Xyloglucan forms gels by the lateral stacking of rod like chains. The 1.5% w/w xyloglucan based in situ gelling formulation showed similar miotic response as shown by 25% w/w Pluronic F127 gel.

The thermoreversible phase transition temperature of poly (N-isopropylacrylamide) (PNIPAAm), a well-known thermosensitive polymer is 32° C. Because of its phase transition temperature closer to human body surface temperature, this in situ gel forming polymer has been utilized.

Addition of methylcellulose, HPMC, CMC, mannitol and sorbitol as viscosity enhancing agents to in situ gelling polymer can be utilized. Thermally sensitive neutral solutions based on chitosan/polyol salt combinations (DOI: 10.1016/s0142-9612(00)00116-2) is also a temperature triggered in situ gelling system that can be used.

The drug loaded in situ gelling system can use ion triggered in situ gelling polymers. These include polymers whose solution viscosity increases upon exposure to ionic concentration of the body fluids such as tear fluids. It is also called osmotically induced gelation. Ion sensitive polymers can crosslink with cations (monovalent, divalant) present in lacrimal fluid on ocular surface and enhance the retention time of drug. Ion triggered in situ gelling polymeric systems include gellan gum which is commercially known as Gelrite®, and alginic acid/sodium alginate: Sodium alginate is a natural hydrophilic polysaccharide approved by FDA for human use as wound dressing material and as food additives consist of (1→4) linked β-D-mannuronic acid (M) and α-L guluronic acid (G) units of varying composition and sequence. Alginate transforms into stable gel upon exposure to divalent cations such as Ca²⁺ in the body. The % of guluronic acid in polymer backbone plays a major role in alginate gelation and drug release. Alginates with guluronic acid contents >65% gelled instantaneously, whereas with low guluronic acid contents gelled slowly and forms weak gels. Ion activated in situ gelation of sodium alginate in combination of other viscosity enhancer such as HPMC can be used. Low contraction of Ca salt can be preloaded into the formulation before injection, which will not cause gelling in vitro but will to help the gelling in vivo, e.g. 0.1%˜0.6% calcium gluconate solution. The amount of Ca salt to be added to help gelling in vivo but not cause gelling in vitro is dependent of the concentration of alginate in the formulation. Higher concentration of alginate requires lower concentration of Ca salt and low concentration of alginate can tolerate higher concentration of Ca salt while maintain the non-gel state in vitro. The suitable amount of Ca salt to be preloaded into the formulation can be determined experimentally easily by adding different amount of Ca salt to the alginate containing formulation and select the highest amount of Ca salt that does not produce un injectable gel in vitro. In some examples, the in situ gelling system matrix is 1% w/v sodium alginate (e.g. VLVG, NovaMatrix, FMC Biopolymers, Drammen, Norway) and 0.3% w/v calcium D-gluconate in the final drug containing formulation of the current invention.

Combination of gelling enhancing agent including polymers having different gelation mechanisms can also be used. To reduce the amount of polymers required for gelation and to get better gels with improved gelling properties combination of two or more polymers with different gelation mechanism can be used for developing in situ drug delivery system. For example, a combination of thermosensitive polymers, methylcellulose or HPMC and pH triggered polymer Carbopol can be used. The former polymers exhibited thermal gelation and the latter pH dependent gelation. The final formulation formed an easy flowing formulation, which reversibly gelled with a sol-gel transition between 25° C. and 37° C. as well as with a pH increase from 4.0 to 7.4. In some examples, 25% (w/v) Pluronics and 30% (w/v) CAP are used. In one example poloxamer+chitosan based in situ gelling system can be used. Poloxamer-chitosan (16:1) system showed optimum gelation temperature 32° C. In one example a combination of pH and ion triggered polymers based in situ gelling systems can be prepared by blending three different polymers namely Carbopol 940, sodium alginate and guar gum. In one example a formulation can consist of 15% Pluronic F127 and 0.1% low molecular weight chitosan. 0.3% and 14% (w/w) concentrations of Carbopol and Pluronic can be used for preparation of in situ gelling formulations. In another example Poloxamer 407 and 188 are used as thermosensitive polymers and Carbopol 1342P NF is used as pH sensitive polymer and the combined solutions formed gels under physiological conditions. In one example ˜15% Pluronic F127 combined it with polymers like HPMC as a viscosity increasing agent or with polymers such as Carbopol 940, xanthan gum, and sodium alginate (high glucuronic acid content) for pH and cation-triggered sol-gel transition can be used. The combination of methylcellulose or HPMC and Carbopol in some examples. In one example concentration of sodium alginate solution for the in situ gelation is 2% w/w and that for Pluronic F127 it is 14% (w/w). In some examples Triblock (TB) polycaprolactone-polyethylene glycol-polycaprolactone [(PCL-PEG-PCL), BAB] and pentablock copolymers (PBCs) polylactic acid (PLA) [(PLA-PCL-PEG-PCL-PLA), CBABC] and [(PEG-PCL-PLA-PCL-PEG), ABCBA] can be used. In one example in situ gelling system is sodium alginate as ion sensitive polymer and methylcellulose as viscosity enhancing agent. In some examples, Polyacrylic acid (Carbopol 940) or hyaluronic acid, Pluronic F127 and gellan gum are used for pH-triggered in situ gelation, thermoreversible gelation and ion activated system, respectively. HPMC is added with Carbopol or hyaluronic acid as viscosity enhancer and in combination of Pluronic F127 for reducing the concentration of Pluronic F127. Gelrite® is used for cation induced gelation (0.6%). In some embodiments, drug loaded thermosensitive PEG-PCL-PEG (PECE) hydrogel by synthesizing PECE block polymers by coupling MPEG-PCL copolymer using IPDI reagent having sol-gel transition as a function of temperature can be used. The formulation containing PECE (30% w/v) aqueous solution exhibited sol-gel transition at 35° C.

Furthermore, drug loaded liposome, emulations including nanoemulsion, suspension, cyclodextrin, micelles, nanoparticles or microparticles can also be incorporated within the in-situ gel. The drug loaded in situ gelling system can use reactive in situ gel as well, which forms hydrogel by crosslinking after mixing two reactive components together. In some embodiments, hydrogel is prepared by simple mixing of glycol chitosan and oxidized alginate aqueous solution, which can be injected right after being mixed together when it is still injectable as complete crosslinking reaction takes time. The polymer (e.g. hyaluronic acid) and crosslinking agent (e.g. H₂O₂, pentasodium tripolyphosphate) can also be co injected (e.g. using a dual syringe type device) to the body to allow crosslinking take place in vivo. In some embodiments, PEG hydrogel is prepared through thiol-maleimide reaction utilizing 4 arms PEG-Mal and 4 arm PEG-SH. In some embodiments, in situ gelling drug delivery system is thiolated poly (aspartic acid) (ThioPASP). In some embodiments, hydrogel is composed of maleimide-modified c-polyglutamic acid (c-PGA-MA) and thiol end-functionalized 4-arm poly (ethylene glycol) (4-arm PEG-SH) such as those in Acta Biomaterialia 86 (2019) 280-290.

Another type of reactive situ gelling system matrix is injectable drug eluting elastomeric polymer (iDEEP), such as those described in doi: 10.1016/j.gie.2011.12.009. For example, poly (ethylene glycol maleate citrate), PEGMC, is dissolved in deionized water (20 wt %), and combined with poly (ethylene glycol diacrylate) (12 wt %), and tetramethylethylenediamine (0.5 wt %) as iDEEP Part A, suitable amount of drug is also loaded in iDEEP Part A. The iDEEP Part B Component (iDEEP-B) is prepared by dissolving ammonium persulfate redox initiator (0.25 wt %) in deionized water. Combining the Part A and B solutions in a 2:1 ratio, respectively, produces iDEEP gels.

Photocrosslinkable agent can also be used to form in situ gel, which is also a reactive matrix and the gelling reaction is triggered by light irradiation. Examples of photocrosslinkable include polyethylene glycol diacrylate (PEGDA) and photocrosslinkable chitosan hydrogel, such as those described in (DOI): 10.1055/s-0028-1103483. PEGDA gels rapidly at room temperature in the presence of a photoinitiator and light (e.g. UV light).

In some embodiments, drug loaded in situ gelling implant/insert can be used. For example, carboxymethylcellulose sodium (CMC) and sodium alginate (ALG) combination can be used as the matrix. In some embodiments, the drug loaded in situ gelling is in chitosan/HPMC based polymer matrix. In some embodiments, the drug loaded injectable gel or nano/micro particles is in biochronomer (tri(ethylene glycol) poly(orthoester), TEG-POE) based polymer matrix. For example, the injectable gel is 80% TEG-POE (MW 6 kDa), ˜19% methoxypoly(ethylene glycol) (MW 550 Da) and 0.1-1% (by weight) drug.

In some embodiments, the drug loaded in situ gelling is in chitosan-calcium alginate gel microsphere based polymer matrix, such as those described in patent number CN1628861A. For example, the matrix can be chitosan-calcium alginate gel microsphere type material, which is composed of calcium alginate gel microspheres optionally covered with chitosan in 0.5-4.0% sodium alginate solution. The particle size of the calcium alginate gel microspheres is between 1-200 μm; the ratio of the chitosan-calcium alginate gel microspheres to the sodium alginate solution is 10:1-10:30 by volume. The drug can be either encapsulated in the microsphere or in the alginate solution phase or both.

Another in situ gelling material can be used in the said formulation is biodegradable water insoluble polymer such as poly(D,L-lactide-co-hydroxymethyl glycolide) (PLHMGA), PLA, PLGA, PCL, PGA, prolifeprospan such as prolifeprospan 20 or PHB. It can be dissolved in biocompatible water miscible organic solvent such as N-methyl pyrrolidone or DMSO as matrix to load the drug, the drug can be dissolved/dispersed in the PGA or PLGA solution (e.g. 10% 50% PLGA in N-methyl pyrrolidone) or two components are combined immediately before injection. In some embodiments, 50:50 lactide/glycolide PLGA or PLGA with lower lactide content can be used, e.g. 10:90 lactide/glycolide PLGA. When this formulation is injected into the body the water miscible organic solvent dissipates and water penetrates into the organic phase. This leads to phase separation and precipitation of the polymer forming a depot at the site of injection as sustained release implant type material. Although it is not a classic hydro gel gelling system, it is still called gelling in the current invention for illustration purpose. Examples can be found in Atrigel™ delivery system and those in doi: 10.1016/j.jconre1.2014.05.057.

Other gelling or high viscosity materials that can be used in the current invention include: RAD16 peptide, collagen, PNIPAAm-g-MC, the polymer in patent number CN102344559A, modified hyaluronic acid sodium gel in patent number CN104086788B, injectable hyaluronic acid/polyethylene glycol hydrogel in patent number CN106519072A, sodium hyaluronate collagen hydrogels in patent number CN107189119A, Pluronic® F127 and Pluronic® F68, PNIPAAm, poly(lactic acid-co-glycolic acid)-poly(ethylene glycol)-poly(lactic acid-co-glycolic acid) (PLGA-PEG-PLGA) hydrogel (e.g. those in International Journal of Pharmaceutics 490 (2015) 375-383), thermosensitive triblock polymer poly-(DL-lactic acidco-glycolic acid) (PLGA)-polyethylene glycol (PEG)-PLGA (e.g. those in DOI: 10.3109/03639041003680826), system containing poloxamer 188/poloxamer 407/carbopol 934/HPMC (e.g. those in dx.doi.org/10.1155/2014/280928), injectable bioresponsive gel depot (e.g. those in DOI: 10.1002/adma.201801527), PVA-TSPBA hydrogels (e.g. those in Sci. Transl. Med. 10, eaan3682,2018), fibrin hydrogel (e.g. those in patent number CN110393699A), thermo gelling polyurethane/PEG block copolymer (e.g. the amine-functionalized ABA block copolymer, poly(ethylene glycol)-poly(serinol hexamethylene urethane), consists of a hydrophobic block (B): poly(serinol hexamethylene urethane) and a hydrophilic block (A): poly(ethylene glycol), e.g. those disclosed in doi: 10.1016/j.biomaterials.2010.09.044); injectable self-healing polymer-nanoparticle (PNP) hydrogel (dodecyl-modified hydroxypropylmethylcellulose (HPMC-C12) combined with poly(ethylene glycol)-b-poly(lactic acid) (PEG-PLA) nanoparticles (NPs), 2 wt % HPMC-C12+10 wt % NP as those in doi.org/10.1021/acscentsci.0c00732); vaccine self-assembling immune matrix made of (RADA)4 synthetic oligopeptide (e.g. those described in DOI:10.1128/CVI.00714-14); thermal-sensitive hydrogel formulated with N-[(2-hydroxy-3-trimethylammonium) propyl] chitosan chloride (HTCC) and α, β-glycerophosphate (α, β-GP) (e.g. those in doi.org/10.1016/j.biomaterials.2011.11.068); poly(d, 1-lactide)-poly(ethylene glycol)-poly(d,l-lactide) (PDLLA-PEG-PDLLA,PLEL) (e.g. those in doi.org/10.1016/j.apmt.2020.100608); the gelling system in patent application number WO2014006215A1; injectable PEG-b-poly(L-alanine) hydrogel (e.g. those in doi:10.7150/thno.30577); injectable chitosan-alginate porous gel in doi: 10.1002/mabi.201800242; and also the agent that has low viscosity at high shear rate (e.g. 100 S⁻¹ reminiscent of the injection process) and high viscosity (preferably >10 times higher) at a low shear rate (e.g. the condition after being injected).

Other agent that has low viscosity at high shear rate and high viscosity at a low shear rate can also be used as matrix in the formulation of the current inventions either alone or together with other in situ gelling matrix. Example of them include materials having exhibiting pseudoplastic viscosity such as those polysaccharide disclosed in WO2013077357A1, such as xanthan gum, carrageenan, gellan gum, guar gum, locust bean gum, Sacran, or a salt thereof. Suitable concentration of these polysaccharide concentration is 0.5 to 5 w/v% and the pH value of the formulation is between 3-8. In one example, 1-2% xanthan gum (KELTROL, CGT, CP Kelco company) is used alone as the pseudoplastic viscosity enhancing agent or in combination with 2% sodium alginate as in situ gelling matrix in the formulation.

Additional examples and procedures of making these in situ gelling matrix can be found DOI: 10.15406/japlr.2016.02.00022, dx.doi.org/10.1016/j.drudis.2013.10.001, doi.org/10.1016/50920-4105(00)00034-6, their related citations and the reference listed within the current invention and can be readily adopted for the current invention.

Liquid solution as used herein refers to solutions, suspensions, emulsions, drops, ointments, liquid wash, sprays, liposomes which are well known in the art. In some embodiments, the liquid solution contains an aqueous pH buffer agent which resists changes in pH when small quantities of acid or base are added. In some embodiments, the liquid solution has an osmolarity close to the physiological osmolarity value, which can be achieved by adding suitable amount of pharmaceutical acceptable excipient to the formulation.

In certain aspect, the current invention and previous applications from the current inventor disclose methods, compositions and regents to treat autoimmune diseases and allergy or to inhibit anti-drug antibody production or to induce antigen specific immune tolerance by applying the combination of antigen and immunosuppressive agent/drug either as a physical mixture or as synthetic conjugate or as nano/micro/macro particles or implant or liposome) in a sustained (extended) release system such as an in-situ gelling system or high viscosity formulation to the subject/patient in need. The term nano/micro particle means the particle is in either nanometer or micrometer range of size (diameter). For example, the nano/micro particle can be in the size range of 50 nm˜100 μm. The macro particle can be in the size range of 100 μm-10 mm. The particles can be made of biodegradable materials such as PLGA or polysaccharide (e.g. alginate).

A physical mixture means that the mixture of antigen and immunosuppressive agent are simply mechanically mixed (e.g. by stirring or blending) together in their original form (e.g. liquid or solid form such as powder or particles without being encapsulated in other nano or micro particles) without any additional process, e.g. by just mixing them in their original form together, or further size reducing process is applied before or after the mechanical mixing (e.g. crashing, grinding, mulling or homogenizing), or dispersed or dissolved separately in same or different type of liquid and then mix, or co-dispersed in liquid, or co-dissolved in solvent (e.g. water), and optional drying process (e.g. spray drying or lyophilization) can be applied with optional further size reducing process. Physical mixture means antigen and immunosuppressive agent are not encapsulated or conjugated together or encapsulated in nano or micro particles. the list of exemplary immunosuppressive drugs can be found at “immunosuppressive drug” article page in wikipedia. The immunosuppressive agent/drug (immunosuppressants) suitable for the current application include but are not limited to, statins; mTOR inhibitors, such as rapamycin or a rapamycin analog (e.g. everolimus, ridaforolimus/deforolimus and temsirolimus) or the second generation of mTOR inhibitors known as ATP-competitive mTOR kinase inhibitors; anti-inflammatory corticosteroid; TGF-β signaling agents; TGF-β receptor agonists; TLR (Toll-like receptor) inhibitors; pattern recognition receptor inhibitors; NOD-like receptors (NLR) inhibitors; RIG-I-like receptors inhibitors; NOD2 inhibitors; histone deacetylase inhibitors such as trichostatin A; corticosteroids; inhibitors of mitochondrial function such as rotenone; P38 inhibitors; NF-κβ inhibitors such as 6Bio, dexamethasone, TCPA-1, IKK VII; adenosine receptor agonists; prostaglandin E2 agonists (PGE2) such as misoprostol; phosphodiesterase inhibitors including phosphodiesterase 4 inhibitor (PDE4) such as rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator- activated receptor agonists; histone deacetylase inhibitors; calcineurin inhibitors; phosphatase inhibitors; PI3 KB inhibitors such as TGX-221; autophagy inhibitors such as 3-methyladenine; aryl hydrocarbon receptor inhibitors; proteasome inhibitor I (PSI); oxidized ATPs; P2X receptor blockers. Immunosuppressants also include IDO, vitamin D3, cyclosporins such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide, siglec ligand such as sialic acid and its derivative including poly sialic acid sialic acid-lipid conjugate. In embodiments, the immunosuppressant may comprise any of the agents provided herein. The immunosuppressant can be a compound that directly provides the immunosuppressive (e.g., tolerogenic) effect on

APCs or it can be a compound that provides the immunosuppressive (e.g., tolerogenic) effect indirectly (i.e., after being processed in some way after administration). Immunosuppressants, therefore, include prodrug forms of any of the compounds provided herein. In some preferred embodiments, the immunosuppressant used is mTOR inhibitor (such as rapamycin or a rapamycin analog) or methotrexate.

The immunosuppressant also includes heme oxygenase-1 (HO-1) inducer such as cobalt protoporphyrin (CoPP), protoporphyrin IX containing a ferric iron ion (heme B) with a chloride ligand (hemin), hematin, iron protoporphyrin or heme degradation products as well as those described in PCT/EP2015/074819. Siglecs (sialic acid-binding immunoglobulin-type lectins) ligand such as sialic acid or its derivatives is also another type of immunosuppressant that can be used in current invention. PD-L1 is also another type of immunosuppressant that can be used in current invention. PD-L1 can effectively inhibit cytotoxic T cell. Fragment or mimic or derivative of PD-L1 that can bind with PD-1 can also be used instead. Other inhibitory ligands that can bind with inhibitory checkpoint receptor (e.g. A2AR, BTLA, CTLA-4, CD 47, KIR, LAG3, TIM-3, VISTA and etc.) such as B7-H3, B7-H4 can also be used instead of PD-L1. Molecule that can promote T/B reg expansion (e.g. cytokine that can stimulate T/B reg expansion such as IL-2 and TGF-β is also another type of immunosuppressant. Different immunosuppressant can be used as a mixture and be used in combination in the current invention.

Immunosuppressant also include nucleic acids that encode the peptides, polypeptides or proteins provided herein that result in an immunosuppressive (e.g. tolerogenic) immune response. In embodiments, therefore, the immunosuppressant is a nucleic acid that encodes a peptide, polypeptide or protein that results in an immunosuppressive (e.g., tolerogenic) immune response. The nucleic acid can be coupled to synthetic nanocarrier. The nucleic acid may be DNA or RNA, such as mRNA. In embodiments, the inventive compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector.

In some embodiments, the immunosuppressant provided herein are conjugated to or fused with an affinity ligand. When both immunosuppressant and affinity ligand are peptide/protein, they can be constructed as a fused protein by genetic engineering and expression, one can be attached to the N or C terminal of another via an optional linker sequence. The affinity ligand can target or bind to an autoimmune disease causing/affected organ or tissue or cell or protein or antigen. The affinity ligand can be full antibody, antibody fragment, antibody mimetic or their derivatives as well as non-protein molecules such as aptamer, examples are disclosed in prior US patent applications by the current inventor. The term antibody in the current application include both full length antibody, antibody fragment, nanobody and their derivatives. The resulting conjugate or fusion protein can be used to treat related autoimmune disease or allergy by administrating it at therapeutically effective amount to the subject in need (e.g. by injection). The resulting organ/tissue/cell/protein/antigen targeting ligand-immunosuppressant conjugate or fusion can shield the disease suffering cells and induce tolerance. For example, collagen II is abundant in cartilage, anti-collagen II scFv-PD-L1 fusion protein or other anti-collagen II Fab-PD-1 agonist fusion or conjugate can be used to treat rheumatoid arthritis, which will coat the cartilage and chondrocyte with PD-L1 or the like to protect them from T cell attack and induce tolerance. In some embodiments, the affinity ligand is antibody. In some embodiments, the antibody is IgG4 or Fc engineered to reduce its ADCC and CDC effect. In some embodiments, the antibody is engineered to have enhanced ADCP effect such as those described in as those disclosed in prior US patent applications by the current inventor. In some embodiments, mTOR inhibitor (e.g. rapamycin, everolimus, ridaforolimus/deforolimus and temsirolimus) is conjugated to the antibody. The protocol to prepare mTOR inhibitor-antibody conjugate can be found in patent application WO2018227018A1 and readily adopted for the current invention. Additional immunosuppressant that can be used in the current inventions can also be found in patent application WO2018227018A1. In some embodiments, calcineurin inhibitor (e.g. cyclosporine or tacrolimus) is conjugated to the antibody. In some embodiments, anti-inflammatory corticosteroid (e.g. dexamethasone or betamethasone) is conjugated to the antibody. In some embodiments, PD-L1 is fused to the antibody. In some embodiments, anti-inflammatory cytokine or its derivative (e.g. IL-2, IL-2-anti IL-2 antibody complex, IL-10, TGF-β is fused to the antibody. In some embodiments, the antibody can bind with autoantigen expressed by the cell such as those described in the later part of the application (e.g. insulin, islet cell autoantigen-2, GAD, IGRP for diabetes treatment). In some embodiments, the antibody can bind with disease affected tissue or cell or organ by binding to the surface marker of these organ/tissue/cell, which is not autoantigen.

When an antibody is to bind with IgE, preferably it only has one antigen binding moiety such as an antibody fragment has one Fab, e.g. Fab of Omalizumab), single-chain variable fragment (scFv), scFv-Fc fusion to avoid IgE clustering on mast cell. It can be conjugated with either immunosuppressant or cytotoxic drug such as those used cancer treating ADC. The resulting conjugate can be used to treat allergy.

In some embodiments, the immunosuppressants provided herein are coupled to synthetic nanocarriers or microcarriers. In preferable embodiments, the immunosuppressant is an element that is in addition to the material that makes up the structure of the synthetic nanocarrier or microcarrier. For example, in one embodiment, where the synthetic nanocarrier or microcarrier is made up of one or more polymers, the immunosuppressant is a compound that is in addition and coupled to the one or more polymers. As another example, in one embodiment, where the synthetic nanocarrier or microcarrier is made up of one or more lipids, the immunosuppressant is again in addition and coupled to the one or more lipids. In embodiments, such as where the material of the synthetic nanocarrier or microcarrier also results in an immunosuppressive (e.g., tolerogenic) effect, the immunosuppressant is an element present in addition to the material of the synthetic nanocarrier or microcarrier that results in an immunosuppressive (e.g., tolerogenic) effect.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD16, anti-CD3; tacrolimus (FK506) and etc. Further immunosuppressants, are known to those of skill in the art, and the invention is not limited in this respect. Additional immunosuppressants can be found in patent and patent applications U.S. Ser. No. 13/880,778, U.S. Ser. No. 14/934,135, CA2910579, U.S. Ser. No. 13/084,662, U.S. Ser. No. 14/269,048, U.S. Pat. No. 8,652,487, WO2012054920A2, WO2016073799A1, WO2012149393 A3, WO2014179771A1, PCT/US2012/035405, US20110262491, U.S. Pat. No. 8,652,487 and other patent application filed by Selecta Biosciences.

Selecta's publications disclose synthetic nanocarrier methods, and related compositions, comprising B cell and/or MEW Class II-restricted epitopes and immunosuppressants in order to generate tolerogenic immune responses. In their disclosure, the antigen/epitope is conjugated to the nanocarrier and immunosuppressants is coupled to the nanocarrier. An alternative method and composition are to use nano/micro particle having antigen/epitope non-covalently adsorbed to its surface and immunosuppressant encapsulated within an in-situ gelling system or high viscosity formulation. The nano/micro particles can be made of biodegradable materials such as PLGA. These kinds of nano/micro particles (e.g. 10 nm˜10 μm of diameter in size) can be given to the patient in need as injection or inhaler to induce immune tolerance. The encapsulation of immunosuppressant is well known to the skilled in the art and can be adopted from related publications readily. The surface of the nano/micro particles can have charged groups such as amino or carboxyl group to increase the binding of antigen/epitope to its surface; it can also have a hydrophobic surface to allow binding antigen/epitope via hydrophobic interaction; or the combination of them. Introducing charged groups to the surface can be done by using surface modification or using amine or carboxyl group containing molecules to prepare the nano/micro particles. The antigen/epitope can also be conjugated with a lipophilic moiety such as lipid molecule such as fatty acid or cholesterol to increase its binding to nano/micro particles. The adsorption of antigen/epitope to the nano/micro particle surface can be done by incubating antigen/epitope with the nano/micro particle (e.g. 4° C. overnight in aqueous solution buffer such as 1×PBS) and then removing the unbound antigen/epitope (e.g. washing the nano/micro particle with aqueous buffer several times, similar to the ELISA plate coating procedure). In one example, 50 nm˜200 nm size PLGA nano particle encapsulated with 10% by weight of rapamycin is prepared according to the literature. Next the PLGA nano particle is mixed with OVA (10 mg/mL) at 4° C. overnight to generate the OVA (ovalbumin) coated particle. The particle is washed 3 times with PBS to remove unbound OVA. In another example, rapamycin is dissolved in DMSO at 50 mg/ml. A total of 50 μL rapamycin is added to 1 ml PLGA (5 mg/ ml) dissolved in dichloromethane. Next the mixture is homogenized with 0.4 ml 5% OVA solution for 10 min using ultrasonication. The o/w emulsion is added to 2.1 ml of a 5% w/v solution of PVA to evaporate the organic solvent for 4 h at room temperature. OVA coated nano particles containing rapamycin are obtained after centrifugation at 3,500 g for 20 min. Additional washing step can be performed to obtain unbound OVA free particles. This OVA coated particle in 2-6% sodium alginate or 0.5-2% crosslinked hyaluronic acid can be given to the target in need to induce OVA immune tolerance, using the similar protocol described in the publications (e.g. those from Selecta Bio). For example, 5 mg-50 mg of the particle in 3% sodium alginate or in 1% crosslinked hyaluronic acid can be injected to a patient with OVA intolerance weekly for 3 times to induce OVA tolerance as subcutaneous or intramuscular injection or intralymphatic injection or being injected proximal to the lymph node. The OVA can be replaced with other antigen/epitope molecule to induce corresponding immune tolerance. In another sample, lipophilic carboxylic acid or lipophilic amine or anionic detergent or cationic detergent (e.g. fatty acid such as caprylic acid, lauric acid; or cationic lipid such as DOTMA, DOTAP, cholesterylamine) can be added to the PLGA to prepare PLGA particle having surface charge. In one example, rapamycin is dissolved in DMSO at 50 mg/ml with lauric acid at 10 mg/mL. A total of 50 μL rapamycin/lauric acid is added to 1 ml PLGA (5 mg/ml PLGA) dissolved in dichloromethane. Next the mixture is homogenized with 0.1 ml 2% caprylic acid solution for 10 min using ultrasonication. The o/w emulsion is evaporated to remove the organic solvent for 4 h at room temperature. The resulting PLGA particle is washed 3 times with PBS and then incubated with OVA to prepare OVA bound particles. In one example, 10 mg˜100 mg of the particle in 2% sodium alginate and 1% HPMC can be injected to a patient 56 with OVA intolerance very month for 3 times to induce OVA tolerance as subcutaneous or intramuscular injection or intralymphatic injection. In another, PLGA rapamycin microparticles are synthesized using PLGA polymer (PLGA, 50:50 or 65:35, molecular weights from 10,000-85,000 Da), using single emulsion method. Briefly, 100 mg PLGA is dissolved in 2 mL dichloromethane (DCM) with 10 mg rapamycin and homogenized in 1% Poly Vinyl Alcohol (10 mL, 87˜89% hydrolysed, MW 13,000˜23,000 kDa, Sigma #363170) at 2000 rpm. This solution is added to 1% PVA 100 mL and is allowed to stir continuously for 3-4 h to evaporate DCM completely. The solution is then centrifuged at 11,000 g and washed using deionized water twice to wash away the excess PVA. The microparticles are then re-suspended in deionized water and rapidly frozen at −80° C. followed by lyophilization. Encapsulation efficiencies 30-50% can be achieved. A sterile suspension of 10 mg/mL microparticle mixed with suitable amount of antigen (e.g. 1 mg/mL OVA as final concentration) in 1× PBS pH 7 or 3% sodium alginate or in 15-25% Pluronic F127 or Poloxamer 407 is injected to treat related antigen intolerance disease.

Furthermore, antigen/epitope can also be encapsulated within the nano/micro particle besides being conjugated or adsorbed to its surface. The preparation of antigen/epitope encapsulation is well known to the skilled in the art and can be adopted from related publications readily, e.g. using a double emulsion water/oil/water system. In one example, 10 g of DL-PLGA (80:20, MW ˜50,000) is dissolved in 50 g of a mixed solvent consisting of 35 wt % acetone and 65 wt % chloroform. 200˜500 mg of rapamycin or rapamycin analogue is added together with 10-50 mg of peptide antigen, and the mixture is stirred vigorously for 30 min. This organic phase is then added slowly to 500 g of 5 wt % aqueous poly(vinyl alcohol) . During the addition of the organic phase, the PVA solution is stirred at 800 rpm to form a stable oil-in-water emulsion. After the emulsion stirred for 10 min, vacuum is applied and the stir rate is lowered to 600 rpm for 20 h to remove the volatile solvents. After centrifugation, the resulting pellet of microcapsules is washed thoroughly with deionized water, and the microcapsules are wet sieved to collect the 30˜80 μm diameter particles. Then dried in a vacuum chamber. maintained at room temperature. The size of the microcapsules can be adjusted by using sieve having different mesh size. A sterile suspension of 50 mg/mL microparticle in 1× PBS pH =7 or 3% sodium alginate or in 15-25% Pluronic F127 or Poloxamer 407 is injected to treat related peptide antigen intolerance disease. Bigger size particles have longer in vivo drug release time.

Rapamycin containing microparticles with optional anti-inflammatory steroid can also be used in the current invention. Those compositions/formulation can also be in a sustained release system such as in-situ gelling system disclosed in the current invention.

US patent application number US20130287729 disclosed antigen-specific, tolerance-inducing microparticles and uses thereof. It disclosed a microparticle (0.5 μm-10.0 μm in size) for targeting an antigen-presenting immune cell of interest and for inducing antigen-specific immune tolerance, wherein the microparticle comprises an antigen and a therapeutic agent wherein the therapeutic agent is an immunomodulatory agent, an immunosuppressive tolerogenic agent, or an agent that recruits the antigen-presenting immune cell of interest, wherein the surface of the microparticle comprises a ligand that targets the antigen-presenting immune cell of interest and the microparticle is made of biodegradable material. A further improvement of this method and composition is to use a either nano/micro particle having the size of 50 nm˜5 μm preferably made of biodegradable materials or those disclosed in application US20130287729, in a sustained release formulation such as in-situ gelling system or high viscosity formulation. In some embodiments, the surface of the nano/micro particle is coated with Fc portion of an antibody or a full antibody with its Fc portion facing outside. This will bind with the FcR to facilitate APC uptake. In other embodiments, the surface of the nano/micro particle needs not to have a ligand that targets the antigen-presenting immune cell. In some embodiments, it can have antigen/epitope coated on its surface. The inner part of the nano/micro particle contains immunosuppressive agent listed in the current application and optionally antigen/epitope, e.g. by encapsulation. The preparation method is well known to the skilled in the art and can be adopted from related publications readily. For example, 0.5 mg˜50 mg of the above particle (5-25% of the formulation) containing gluten and rapamycin in 3% sodium alginate with optional 0.5-2% HPMC, or in 15-25% Pluronic F127 or in 15-25% Poloxamer 407 can be injected to a patient with gluten intolerance monthly for 3 times to induce gluten tolerance as subcutaneous or intralymphatic injection.

US patent application 20160338953 disclosed a liposome-based immunotherapy. It provided a liposome encapsulating an autoantigen, wherein the liposome has a size comprised from 500 to 15000 nm and the liposome membrane comprises phosphatydilserine (PS) in an amount comprised from 10 to 40% by weight with respect to the total membrane liposomal composition. Pharmaceutical or veterinary compositions comprising a therapeutically effective amount of said liposome were also provided. Further, it provided liposomes and pharmaceutical or veterinary compositions as defined above for use as a medicament, particularly for the treatment of autoimmune diseases. Finally, it provided liposomes and pharmaceutical or veterinary compositions as defined above for use in the restoration of tolerance to self in a patient suffering from an autoimmune disease. The current invention also discloses antigen-specific, tolerance-inducing liposome and uses thereof. The liposome contains immunosuppressive agent listed in the current application (and optionally antigen/epitope molecule) inside by encapsulation. Optionally the surface of the liposome can also have antigen/epitope coated. It can be given to the patient in need as injection to induce immune tolerance. The lipid used for liposome can include but not limited to phosphatydilserine at 10 to 40% by weight of the membrane. It can also use non-phosphatydilserine lipid to prepare the membrane. The antigen/epitope can also be conjugated with a lipid type molecule such as fatty acid or phospholipid or cholesterol derivative to allow it to be inserted to the liposome membrane. Suitable liposome can have a size between 50 nm˜20 μm. The preparation method and the protocol of its use are well known to the skilled in the art and can be adopted from related publications readily such as those in US20160338953. Example of the lipid molecule suitable for the current invention to prepare liposome includes but is not limited to phospholipid, glycerolipid, glycerophospholipid, sphingolipid, ceramide, glycerophosphoethanolamine, sterol or steroid. These lipid molecules can also be used to prepare the antigen/epitope-lipid conjugate. Membrane anchoring peptide-antigen/epitope conjugate can also be used instead of antigen/epitope-lipid conjugate. In addition, other molecule that can promote TB reg expansion (e.g. IL-2 and/or TGF-β and PD-L1) can also be coated/conjugated to and/or encapsulated within the liposome and nano/micro particle. These liposomes as well as the liposome in patent application US 20160338953 can be in a sustained release formulation such as in-situ gelling system or high viscosity formulation. For example, 0.5 mg˜50 mg of the said liposome of the current invention (5-25% of the formulation) containing egg white antigen such as ovomucoid and rapamycin in 3% sodium alginate with optional 0.5-2% HPMC, or in 15-25% Pluronic F127 or in 15-25% Poloxamer 407 can be injected to a patient with egg white intolerance monthly for 3 times to induce egg white tolerance as subcutaneous or intramuscular injection or intralymphatic injection at inguinal lymph node.

Current invention discloses novel reagents and compositions comprising antigen and immunosuppressant in a sustained release formulation such as in-situ gelling system or high viscosity formulation. Those novel reagents and formulations can be given as either subcutaneous injection or intramuscular injections or intradermal injections injection at pharmaceutical effective amount to treat autoimmune disease or allergy or inhibit anti-drug antibody production or induce antigen specific immune tolerance in a subject. Furthermore, those reagents and compositions can also be injected into lymph node (e.g. inguinal lymph node) instead for the same purpose. Intralymphatic allergen administration is known and the same procedure can be readily adopted for the current invention. The reagents and formulations disclosed in said prior applications by the current inventor can also be used as intralymphatic injection. Molecule that can promote T/B reg expansion and/or inhibit harmful auto reactive T/B cell (e.g. IL-2, TGF-β, PD-L1, IL-15, IL-10, IL-21, IL-27, IL-2/anti-IL-2 antibody complexes or their mimics or derivatives such as a pegylated IL-2 NKTR-358) can also be co-injected or included in the formulation to be injected intralymphaticly. The reagents and formulations in the said previous applications and current invention by the current inventor contains disease specific antigen such as B cell antigen, T cell antigen in MHC-peptide complex form or the antigen epitope, mimotope, peptide (or its derivative) of T cell antigen that can bind with MHC to form the MHC-peptide complex. Instated of using antigen directly in the said reagent, composition or formulation, nucleic acid encoding these antigen/epitope can also be used instead such as mRNA encoding them. The mRNA can be in a delivery system such as liposome or lipid vector and can also be modified to improve the target expression using well know methods and protocol. In some embodiments, the amount of the reagent or composition injected into lymph node is between 0.01 mg˜50 mg of drug with injection volume between 0.1 ml to 1 ml per lymph node such as 1 mg monthly or bi weekly for 3 months to induce the antigen specific immune tolerance.

The immunosuppressive agent can be in the form of active agent, prodrug form, micro particle or nano particle form or liposome form. The antigen can be either B cell antigen/epitope or T cell antigen/epitope (e.g. MHC-peptide complex or conjugate; or the peptide antigen that can bind with MHC) or their combination. The combination can be either B cell antigen/epitope with T cell antigen/epitope; or the combination of several different B cell antigen/epitope and/or several different T cell antigen/epitope targeting the same disease or different diseases. The use of peptide antigen (T cell epitope) that can bind with MHC to form MHC-peptide complex in vivo (T cell antigen) instead of the peptide-MHC complex reduce the size and molecular weight, which can improve the transdermal delivery. Examples of them can be found in the prior and current applications and related publications.

Human MHC class I and II are also called human leukocyte antigen (HLA). The most studied HLA genes are the nine classical MHC genes: HLA-A, HLA-B, HLA-C, HLA-DPA1, HLA-DPB1, HLA-DQA1, HLA-DQB1, HLA-DRA, and HLA-DRB1. In humans, the MHC gene cluster is divided into three regions: classes I, II, and III. The A, B and C genes belong to MHC class I, whereas the six D genes belong to class II. There are also non-classical MHC in human. Peptide or peptide MHC complex (pMHC) suitable for the current invention can be found from prior arts and publications readily. The peptide and MHC in the peptide MHC complex can be either covalently conjugated (or expressed) together or bound together to form a non-covalent complex. There are many autoimmune diseases related peptide MHC complex in human or animal being identified. For example, patent applications US20170095544, US20180127481, US20090155292 and US20150125536 disclosed disease specific peptide MHC complex, which can be really adopted for the current application. The MHC class I component can comprise all or part of a HLA-A, HLA-B, HLA-C, HLA-E, HLA-F, HLA-G molecule, particularly all or part of a HLA-A molecule, such as a HLA-A*0201 MHC class I molecule. The non-classical MHC class I component can comprise CD1-like molecules. An MHC class II component may comprise all or part of a HLA-DR, HLA-DQ, or HLA-DP. In certain aspects to treat autoimmune disease and allergy, the antigen/MHC complex is covalently or non-covalently coupled or attached to a substrate (antigen/MHC/particle complex or antigen/MHC/linear polymer). As used herein and unless specifically noted, the term MHC in the context of an pMHC complex intends a classical or a non-classical MHC class I protein and/or or classical or non-classical MHC class II protein, any loci of HLA DR, HLA DQ, HLA DP, HLA-A, HLA-B, HLA-C, HLA-E, CD1d, or a fragment or biological equivalent thereof, dual or single chain constructs, dimers (Fc fusions). In certain embodiments, the MHC class 1 component may comprise, consist essentially of, or alternatively further consist thereof all or part of a HLA-A, HLA-B, HLA-C, HLA-E, HLA-F, HLA-G or CD-1 molecule. In embodiments wherein the MHC component is a MHC class II component, the MHC class II component may comprise, consist essentially of, or alternatively further consist thereof all or a part of a HLA-DR, HLA-DQ, or HLA-DP. In certain embodiments, the MHC may comprise HLA DRB1, HLA DRB3, HLA DRB4, HLA DRB5, HLA DQB1, HLA DQA1, IAg7, I-Ab, I-Ad, HLA-DQ, HLA-DP, HLA-A, HLA-B, HLA-C, HLA-E or CD1d. Non-classical MHC molecules are also contemplated for use in MHC complexes of the disclosure. In some embodiments, non-classical MHC molecules are non- polymorphic, conserved among species, and possess narrow, deep, hydrophobic ligand binding pockets. These binding pockets are capable of presenting glycolipids and phospholipids to natural killer T (NKT) cells. NKT cells represent a unique lymphocyte population that co-express NK cell markers and a semi-invariant T cell receptor (TCR). They are implicated in the regulation of immune responses associated with a broad range of diseases.

The T cell recognize T cell antigen by its TCR receptor. The T cell antigen normally is in the form of MHC-epitope binding complex. The epitope normally is a peptide (sometimes other molecules such as carbohydrate) processed by APC. In the current invention, the antigen for T cells can be the formed MHC-epitope complex or its fragment/derivatives/mimics, which has higher specific affinity to TCR than the epitope alone. It can be the monomer form or oligomer (dimer, trimer, tetramer, pentamer or even higher degree polymer) form such as the MHC tetramer currently used in research to label immune cells. For example, HLA-A2insB10-18 tetramer (e.g. those in doi: 10.1073/pnas.0508621102) can be conjugated with the cell inactivating agent with an optional linker to treat Type 1 diabetes in human by inactivating the autoimmune T cell. The epitope (e.g. peptide) can be covalently conjugated with MHC to increase its stability by well-known means as disclosed in well-known publications. Similarly, the antigen used for B cell in the current invention can also be oligomer or polymer form. However, sometimes the antigen used for B cell inactivation do not require the MHC component.

In some embodiments, the autoimmune disease-relevant antigens are:

one or more diabetes-relevant antigens and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof, and their combinations;

one or more multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof, and their combinations;

one or more Celiac Disease-relevant antigen and is derived from gliadin or a fragment or an equivalent thereof, and their combinations, and their combinations;

one or more primary biliary cirrhosis-relevant antigen and is derived from PDC-E2 or a fragment or an equivalent thereof, and their combinations;

one or more pemphigus folliaceus-relevant antigen and/or pemphigus vulgaris-relevant antigen and is derived from an antigen selected from one or more of the group: DG1, DG3, or a fragment or an equivalent of each thereof, and their combinations;

one or more neuromyelitis optica spectrum disorder-relevant antigen and is derived from AQP4 or a fragment or an equivalent thereof, and their combinations;

one or more arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof, and their combinations;

one or more allergic asthma-relevant antigen and is derived from an antigen selected from one or more of the group: DERP1, DERP2, or a fragment or an equivalent of each thereof, and their combinations;

one or more inflammatory bowel disease-relevant antigen and is derived from an antigen selected from one or more of the group: flagelin, Fla-2, Fla-X, YIDX, bacteroides integrase, or a fragment or an equivalent of each thereof, and their combinations;

one or more systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, RO60, RO52, histones, or a fragment or an equivalent of each thereof, and their combinations;

one or more atherosclerosis-relevant antigen and is derived from an antigen selected from one or more of the group: ApoB, ApoE or a fragment or an equivalent of each thereof, and their combinations; one or more COPD-relevant antigen and/or emphysema-relevant antigen and is derived from elastin or a fragment or an equivalent thereof, and their combinations;

one or more psoriasis-relevant antigen and is derived from an antigen selected from one or more of the group: Cap18, ADMTSL5, ATL5, or a fragment or an equivalent of each thereof, and their combinations; p one or more autoimmune hepatitis-relevant antigen and is derived from an antigen selected from one or more of the group: CYP2D6, SLA, or a fragment or an equivalent of each thereof; and their combinations;

one or more Sjogren's Syndrome-relevant antigen and is derived from an antigen selected from one or more of the group: (SS-A)/Ro, (SS-B)/La, MR3, RO60, RO52, or a fragment or an equivalent of each thereof; and their combinations;

one or more scleroderma-relevant antigen and is derived from an antigen selected from one or more of the group: CENP-C, TOP 1, RNA polymerase III, or a fragment or an equivalent of each thereof, and their combinations;

one or more anti-phospholipid syndrome-relevant antigen and is derived from APOH or a fragment or an equivalent thereof, and their combinations; one or more ANCA-associated vasculitis-relevant antigen and is derived from an antigen selected from one or more of the group: MIPO, PRTN3, or a fragment or an equivalent of each thereof, and their combinations;

one or more Stiff Man Syndrome-relevant antigen and is derived from GAD or a fragment or an equivalent thereof.

Examples of the sequence of these antigen peptides can be readily found in patent applications US20170095544, US20090155292 and other prior arts. For example, diabetes-relevant antigens include but are not limited to those derived from PPI, IGRP, GAD, islet cell autoantigen-2 (ICA2), and/or insulin. Autoreactive, diabetes-relevant antigenic peptides include, but are not limited to, include those listed in the following, in addition to the peptides and proteins disclosed in US patent US10124045B2, as well as equivalents and/or combinations of each thereof. For example, they can be the antigens disclosed in U.S. patent US10124045B2 as below: GAD65114-123, GAD65536-545, GFAP143-151, GFAP214-222, IA-2172-180, IA-2482-490, IA-2805-813, ppIAPPs5i3, ppIAPP9-17, IGRP152-160, IGRP211-219, IGRP215-223, IGRP222-230, IGRP228-236, IGRP265-273, IGRP293-301, proinsulinL2-10, proinsulinL3-11, proinsulinL6-14, proinsulinB5-14, proinsulinB10-18, proinsulinB14-22, proinsulinB15-24, proinsulinB17-25, proinsulinB18-27, proinsulinB20-27, proinsulinB21-29, proinsulinB25-C1, proinsulinB27-05, proinsulinC20-28, proinsulinC25-33, proinsulinC29-A5, proinsulinA1-10, proinsulinA2-10, proinsulinA12-20, hInsB10-18, hIGRP228-236, hIGRP265-273, IGRP206-214, hIGRP206-214, NRP-A7, NRP-I4, NRP-V7, YAI/Db, INS B15-23, PPI76-90 (K88S), IGRP13-25, GAD555-567, GAD555-567(5571), IGRP23-35, B24-C36, PPI76-90, INS-I9, TUM, G6Pase.

In certain aspects, the human disease and disease related pMHC complex used for the treatment can be:

type I diabetes and the pMHC complex is selected from the group of: insB10-18- HLA-A2 , PPI76-90(K88S)-HLA-DRB1*0401/DRA, IGRP13-25-HLA-DRB1*0301/DRA, GAD555-567-HLA-DRB1*0401/DRA, GAD555-567(557I)-HLA-DRB1*0401/DRA, IGRP23-35-HLA-DRB1*0401/DRA, B24-C36-HLA-DRB1*0301/DRA, or PPI76-90-HLA-DRB1*0401/DRA;

multiple sclerosis and the pMHC complex is selected from the group of: MBP86-98-HLA-DRB1*1501/DRA, MBP89-101-HLA-DRB5*0101/DRA, MOG38-52-HLA-DRB4*0101/DRA, MOG97-109(E107S)-HLA-DRB1*0401/DRA, MOG203-217-HLA-DRB3*0101/DRA, PLP54-68-HLA-DRB3*0101/DRA, PLP94-108-HLA-DRB1*0301/DRA, PLP250-264-HLA-DRB4*0101/DRA, MPB13-32-HLA-DRB5*0101/DRA, MPB83-99-HLA-DRB5*0101/DRA, MPB111-129-HLA-DRB5*0101/DRA, MPB146-170-HLA-DRB5*0101/DRA, MOG223-237-HLA-DRB3*0202/DRA, MOG6-20-HLA-DRB5*0101/DRA, PLP88-102-HLA-DRB3*0202/DRA, or PLP139-154-HLA-DRB5*0101/DRA;

Celiac Disease and the pMHC complex is selected from the group of: aGlia57-68-HLA-DQA1*0501/HLA-DQB1*0201, aGlia62-72-HLA-DQA1*0501/HLA-DQB1*0201, aGlia217-229-HLA-DQA1*0501/HLA-DQB1*0302, or aGlia217-229-HLA-DQA1*03/HLA-DQB1*0302;

primary biliary cirrhosis and the pMHC complex is selected from the group of: PDC-E2122-135-HLA-DRB4*0101/DRA, PDC-E2249-262-HLA-DRB4*0101/DRA, PDC-E2249-263-HLA-DRB1*0801/DRA, PDC-E2629-643-HLA-DRB1*0801/DRA, PDC-E272-86-HLA-DRB3*0202/DRA, PDC-E2353-367-HLA-DRB3*0202/DRA, PDC-E2422-436-HLA-DRB3*0202/DRA, PDC-E2629-643-HLA-DRB4*0101/DRA, PDC-E280-94-HLA-DRBS*0101/DRA, PDC-E2353-367-HLA-DRB5*0101/DRA, or PDC-E2535-549-HLA-DRB5*0101/DRA, mPDC-E2166-181-I-Ag7, or mPDC-E282-96-I-Ag7;

neuromyelitis optica spectrum disorder and the pMHC complex is selected from the group of: AQP4284-298-HLA-DRB1*0301/DRA, AQP463-76-HLA-DRB1*0301/DRA, AQP4129-143-HLA-DRB1*0401/DRA, or AQP439-53-HLA-DRB1*1501/DRA;

allergic asthma and the pMHC complex is selected from the group of: DERP-116-30-HLA-DRB1*0101/DRA, DERP-116-30-HLA-DRB1*1501/DRA, DERP1171-185-HLA-DRB 1*1501/DRA, DERP-1110-124-HLA-DPB1*0401/DRA, DERP-226-40-HLA-DRB1*0101/DRA; DERP-226-40-HLA-DRB1*1501/DRA, or DERP-2107-121-HLA-DRB1*0301/DRA.

The liquid and solution in the current invention are aqueous solution unless specified. The drug 80 (e.g. TLR agonist, antigen, immunosuppressant) in the liquid formulation can be either in the form of solubilized drug or insoluble form such as aggregate, particles including crystals and precipitations. In some embodiments, the drug in the liquid form is present as suspension. Some drug such imiquimod, rapamycin has low water solubility, they can be present in the liquid form as fine particle suspensions. Additional aqueous solubility-enhancing excipient can be added to the formulation to improve the solubility of poorly water soluble drug, such as suitable amount of surfactant (e.g. 0.05%˜0.5% tween-20, tween-60, tween-80, lecithin, spans, fatty acid esters of glycerol, alkyl polyglucosides), polymers (e.g. 0.2-2% PVA, 1%-10% PEG), organic solvent as co-solvent (e.g. 2-20% ethanol, DMSO, propylene glycol).

A biodegradable implant encapsulating antigen and immunosuppressant, or a biodegradable implant encapsulating antigen and adjuvant type agent (e.g. TLR agonist), or a biodegradable implant encapsulating antigen only can also be used to induce tolerance for the antigen to treat the antigen related diseases such as allergy. The size of the implant can be bigger than 10 μm in diameter, preferably >100 μm, if the implant is a macro particle. For example, a 2 mm (length)×0.3 mm (diameter) rod made with PLGA containing 0.5 μg-0.5 mg gliadin and optional 1-3 mg rapamycin (or TLR agonist such as 0.1-1 mg imiquimod or 0.5-5 mg poly IC or 0.5-5 mg CPG ODN) and can be used as an implant underneath the skin to treat gluten intolerance. Other implant format can also be used such as NanoPortal Capsule™ from Nanoprecision Medical and Medici Drug Delivery System™ from Intarcia, as long as they can deliver the antigen and optional immunosuppressant or optional adjuvant type agent simultaneously and continually over time as a sustained delivery system. Macroscale drug delivery systems such as mesoporous silica micro-rod scaffolds can also be used instead as the sustained release system. Other therapeutically safe and effective amount of allergen such as pollen extract, dust mite extract, other food allergen can also be used instead of gliadin.

In one example, a composition and liquid formulation contains 0.5 ug-0.5 mg gluten/mL (e.g. G5004 gluten from wheat, Sigma) in 2-5% sodium alginate pH=7 for gluten intolerance treatment. In one example, a composition and liquid formulation contains 0.5 ug-0.5 mg gluten/mL (e.g. G5004 gluten from wheat, Sigma) and 0.05 mg-5 mg/mL of rapamycin or 0.1 mg-0 mg/mL methotrexate in 2-5% sodium alginate for gluten intolerance treatment, optional solubility enhancing excipient such as 0.1% tween-20 or 5% propylene glycol can also be incorporated in the formulation. It can further contain 200-2000 IU/mL IL-2/anti-IL-2 antibody complexes or their mimics or derivatives such as a pegylated IL-2 NKTR-358. The initial injection dose can be the maximal tolerable dose, e.g. 0.5 mL formulation containing 10 μg/mL gluten for people can tolerate 5 μg of gluten injection. That is, the treatment involves a series of dose or formulations, the first dose or formulation contains lowest amount of allergen and it gradually increases over time in the later dose or formulation while the amount of other drug (e.g. immunosuppressant or immune enhancing agent) can be unchanged. The allergen amount in the first formulation can be the highest amount of allergen that can be tolerated by patient without causing severe allergenic reaction. When the tolerance builds up, injection volume and/or antigen concentration can be increased, similar to the standard allergen desensitization treatment method. Antigen such as gluten and immunosuppressant such as rapamycin and/or methotrexate in in-situ gelling formulation can be in dry form such as lyophilized powder/cake with optional bulking agent/lyoprotectant (e.g. 2-5% sucrose) instead of liquid, those components can be simply mixed together physically, they can also be co-dissolved and then dried and then placed in a vial. In one example, 1-1000 μg gluten and 1.7-2 g poloxamer 407 are mixed in 10 mL water vigorously for 10 min and then lyophilized, and then the dry mixture can be reconstituted with water before injection. In another example, 1-1000 μg gluten and 1 mg of rapamycin, 1.7-2 g poloxamer 407 are mixed in 10 mL water vigorously for 10 min and then lyophilized, and then the dry mixture can be reconstituted with 10 mL water before injection. 1 mg of rapamycin in this formulation can be replaced 1 mg polyIC or 0.25 mg imiquimod instead to treat allergy. In another example, a liquid formulation contains 5 mg/mL methotrexate or 2 mg/mL rapamycin, 5-50 μg/mL gluten (e.g. G5004 gluten from wheat, Sigma) in PGA or PLGA solution (e.g. 30%-50% PLGA in N-methyl pyrrolidone or DMSO). This can be injected to induce gluten tolerance and treat gluten intolerance. The gluten can be replaced with gliadin instead.

To treat allergy to egg, the gluten in the above examples can be replaced with egg white protein such as to reach a final concentration of 0.1 μg˜-0.5 mg/mL of ovomucoid (Gal d 1) or 0.1 μm˜0.5 mg/mL ovalbumin (Gal d 2) or their combination with optional 0.1 ug-0.5 mg/mL ovotransferrin (Gal d 3) and 5 ug-5 mg/mL lysozyme (Gal d 4) to treat egg white allergy. In one example, the antigen is peanut antigen ara h2 at 2 μg/mL in 2-5% sodium alginate pH7 or 17-20% poloxamer 407 solution pH7 to treat peanut allergy as injection. In another example, the antigen is peanut antigen ara h2 at 2 μg/mL and 0.2-1 mg/mL of rapamycin is in 2-5% alginate or 17-20% poloxamer 407 solution to treat peanut allergy as injection. In one example, peanut antigen ara h2 0.1 ug˜10 ug, 0.1-2 mg of rapamycin or 0.1-1 mg imiquimod or 0.1-1 mg poly IC in 1 mL 50% PLGA N-methyl pyrrolidone or DMSO solution is used as injection to treat peanut allergy. The peanut antigen ara h2 can also be replaced with peanut protein extract containing mixtures of proteins such as defatted peanut powder. In another example, an injection to treat lupus contains DNA antigen as shown in FIG. 3 of US patent application 16/029,594, which is the double strand DNA (1 mg˜10 mg/mL), and 0.3˜3 mg/mL of rapamycin or fujimycin or temsirolimus in 2-3.5% sodium alginate with 1% HPMC or in 25% Pluronic F127 or 20% Poloxamer 407, or 45% 50:50 lactide/glycolide in N-methyl pyrrolidone or DMSO.

Other pharmaceutically acceptable amount of antigen and immunosuppressant (or adjuvant such as TLR agonist) can also be used in the formulation, as long as it can produce satisfactory biological and therapeutical (e.g. immune tolerance) effect, which can be determined experimentally by screening and testing with well-known protocol and methods. If the antigen is an allergen such as pollen extract, dust mite extract, animal hair extract or nut protein, the amount of the antigen in the formulation can be determined experimentally using allergy skin test, the highest amount of allergen in the formulation can be tolerated in the skin test will be used in the formulation.

The sustained delivery of both antigen and immunosuppressive drug will be uptaken by APC, induce/activate tolerogenic dendritic cell and Treg/Breg, inhibit B cell activation/antibody production, germinal center formation and antigen-specific hypersensitivity reactions, resulting in long term antigen specific immune tolerance.

Current invention discloses methods and regents to treat autoimmune diseases and allergy or to inhibit anti-drug antibody production or to induce antigen specific immune tolerance by applying the mixture of said antigen and said immunosuppressive agent/drug in a sustained release formulation as injection or implant to the object/patient in need. Current invention also discloses methods and regents to treat allergy by applying the mixture of said antigen and vaccine adjuvant type agent in a sustained release formulation as injection or implant to the object/patient in need, said method and said mixture is not intended to treat autoimmune disease and not to inhibit anti-drug antibody. The injection can be given as either subcutaneous injection or intramuscular injections or intradermal injections or intralymphatic injection. The injection can contain a viscosity enhancing agent to increase its viscosity or becomes a gel after being injected, which acts as a sustained release formulation of both antigen and immunosuppressive agent. Molecule that can promote TB reg expansion (e.g. IL-2 and/or TGF-β and/or PD-L1) can also be added into the injection in combination with other immunosuppressive agent. Antigen and immunosuppressive agent can be either in free molecule form or in nano/micro particle from including liposome form.

In certain embodiments, the injection has a viscosity greater than 10,000 cps at room temperature. In certain embodiments, the injection has a viscosity greater than 100,000 cps at room temperature. In certain embodiments, the injection has a viscosity greater than 1,000,000 cps at room temperature. In certain embodiments, the injection has a viscosity of 10,000,000 cps at room temperature. Example of the viscosity enhancing agent can be found readily from known pharmaceutical acceptable excipient such as hyaluronic acid, starch and carbomer. In some embodiments, the viscosity enhancing agent is biodegradable. In one example, a viscous injection contains 0.1-100 μg/mL gluten (e.g. G5004 gluten from wheat, Sigma) and 0.2-2 mg/mL of rapamycin or 0.5-5 mg/mL methotrexate and suitable amount of hyaluronic acid (e.g. 20-50 mg/mL) to reach a viscosity of 300,000 cps with optional 1 mg/mL IL-2. The hyaluronic acid can be crosslinked to extend their in vivo half-life. The injection formulation can also be a thermal phase changing formulation. Thermal phase changing formulation is a formulation that change its phase from liquid at low temperature or room temperature (25C) to semisolid/gel when temperature increases to body temperature (37C), which can use the temperature triggered in situ gelling system such as poloxamer as excipient. A thermal phase changing injectable formulation containing both antigen and immunosuppressive agent can be given as either subcutaneous injection or intramuscular injections or intradermal injections to induce antigen specific immune tolerance and treat corresponding autoimmune diseases or allergy. It has low viscosity at low or room temperature but high viscosity at body temperature. The preparation of this kind of thermal phase changing injectable formulation can be adopted from related publications readily by the skilled in the art. For example, a composition of a thermal phase changing injectable formulation is 15 μg/mL gluten (e.g. G5004 gluten from wheat, Sigma) and 3 mg/mL in 25% (w/w) Poloxamer-407 pH7 solution, which can be injected to a patient with gluten intolerance 0.5-1 mL bi-weekly for 3 times to induce gluten tolerance as subcutaneous or intralymphatic injection.

The immunosuppressive agent can also be conjugated to carbohydrate polymer or other bio compatible polymer (e.g. dextran or heparin or hyaluronic acid or poly peptide) to form prodrug as described in U.S. patent application Ser. No. 15/723,173, Ser. No. 16/380,951 and Ser. No. 16/029,594. The novel prodrugs can be in the form of carbohydrate (or other polymer) drug conjugate in which the drug is conjugated to the carbohydrate (or other polymer) with cleavable linkage. More than one drugs can be conjugated to the polymer backbone. Suitable carbohydrate includes sialic acid containing polymer, hyaluronic acid, chondroitin sulfate, dextran, carboxyl dextran, cellulose, carboxyl cellulose and their derivatives. It can also be a linear polymer backbone (e.g. dextran or synthetic polymer such as PVA, PAA). Furthermore, the immune suppressive drug can also be directly conjugated to antigen or conjugated to the antigen via a linker or carrier and used in the formulation. The carrier can be a polymer. For example, the poly sialic acid-rapamycin in FIG. 8 of U.S. patent application Ser. No. 15/723,173 can be used to conjugate to the protein's lysine with EDC coupling (e.g. gluten or antibody drug or gliadin or is peanut antigen protein ara h2) and be used in the formulation (e.g. 100 μg˜15 mg) instead of the mixture of antigen and drug.

The formulation or implant of the current and said previous applications by the current inventors can contain either antigen+drug or antigen-drug conjugate or encapsulated antigen/drug (e.g. in microsphere or nano particle or liposome) or their combinations. The antigen can be either in the form of crude antigen (e.g. peanut extract, gluten, egg white powder, pollen extract, dust mite extract) or purified antigen (e.g. peanut antigen protein ara h2, gliadin) or antigen-drug conjugate or encapsulated antigen (e.g. in microsphere or liposome) or their mixture.

When liposome is used, either the drug or both the antigen and immune suppressive drug can be encapsulated in the liposome. Dendritic cell is abundant in skin, adding DC regulating drug with antigen/allergen in the formulation can be effective to induce tolerance. When liposome expressing both antigen and siglec ligand is used (e.g. those described in the current invention and those in J Clin Invest. 2013 Jul; 123(7): 3074-83, J Immunol. 2013 Aug. 15; 191(4): 1724-31 and US patent U.S. Pat. No. 9,552,183), the liposome can further encapsulate immunosuppressive drug such as rapamycin.

For example, each liposome particle can contain pharmaceutical effective amount of rapamycin (e.g. 1%˜50% liposome weight of rapamycin). This will further increase the efficacy to induce immune tolerance and treating autoimmune diseases/allergy.

Another format suitable for the current application is to use microsphere (microparticle). The term microsphere includes particles from nano meter size to micrometers (e.g. 50 nm˜50 μm in diameter). Preferably the microsphere is biodegradable (e.g. made of biodegradable polymer such as PLGA). For example, the microsphere is made of biodegradable synthetic polymer such as PLGA and immunosuppressive drug such as rapamycin (e.g. 1%˜80% weight of the microsphere) is encapsulated. The size of the microsphere is 3 μm or 300 nm. Antigen is also conjugated to the surface of the microsphere directly or with a linker. Alternatively the antigen is encapsulated in the microsphere. Alternatively, the drug (immunosuppressant) can be conjugated to the surface of the microsphere instead of being encapsulated. Examples of microsphere or antigen-immunosuppressant conjugate suitable for the current application can be readily adopted from the disclosure in the publications such as those in patent application U.S. Ser. No. 13/880,778, U.S. Ser. No. 14/934,135, CA 2910579, U.S. Ser. No. 13/084,662 and US patent U.S. Pat. No. 8,652,487 and other patent application filed by Selecta Biosciences. It can be used to treat autoimmune disease or allergy or to induce immune tolerance, which can be either injected or implanted (being encapsulated inside the implant) or applied topically to the patient. The pharmaceutically acceptable amount of microsphere or conjugate in pharmaceutically acceptable sustained release formulation such as in-situ gelling matrix can be used, as long as it can produce satisfactory therapeutical (e.g. immune tolerance) effect, which can be determined experimentally by screening and testing with well-known protocol.

The sustained delivery formulations containing the combination of antigen and immune suppressant agent are used for allergy, autoimmune diseases and antidrug antibody treatment. When the immune suppressant agent in the embodiments in the current application, example and method is replaced with immune enhancing agent (e.g. vaccine adjuvant such as TLR agonist or

STING agonist) and the antigen is a pathogen antigen or tumor antigen, the transdermal delivery system becomes a vaccine or booster for related pathogen or tumor. For example, the sustained delivery formulations is an in-situ gelling formulation containing co-formulated immune enhancing agent together with pathogen antigen. It can also be an implant form as described previously. Vaccine adjuvant type molecule such as TLR agonists can be used in the current invention such as MPLA, CpG ODNs, imiquimod, poly IC, resiquimod, gardiquimod, R848 and 3M-052. Examples of the pathogen antigen can be either synthetic or purified or the mixture of pathogen antigen. For example, it can be HIV gp-120, it can be flu neuraminidase, it can be the flu virus lysate, it can be HBV surface antigen and it can be tumor cell lysate. Using these antigens will generate immune response against the pathogen or tumor as a vaccine or booster. In some embodiments, the sustained release vaccine formulation is a liquid containing 10 μg˜1 mg antigen such as pathogen antigen or tumor antigen, 50 μg˜5 mg TLR agonist in each dose or each mL of injection, in a sustained release matrix such as said in-situ gelling system described previously. For example, the in-situ gelling system can be 2-9% sodium alginate with optional gelling enhancing agent such as 1% HPMC and optional solubility enhancing excipient such as 0.1% tween-20 or 5-15% DMSO can also be incorporated in the formulation; or a temperature triggered in situ gelling polymers such as 17-25% Pluronic F127 or Poloxamer 407; or 40-50% PLGA in DMSO or N-methyl pyrrolidone. In one example, the formulation contains 100 μg˜1 mg/mL Flu virus lysate, 0.2-2 mg/mL imiquimod or 0.2-2 mg/mL poly IC, and 1 mg/ml cetirizine in 3.5% sodium alginate with optional 1-2 HPMC. In another example, the vaccine formulation contains 20˜100 μg/mL HBV surface antigen, 0.5-10 mg/mL CPGODN1018 adjuvant in 20% Poloxamer 407. It can be injected to generate immunity against HBV as either subcutaneous injection or intramuscular injections or intradermal injections or intralymphatic injection. In another example, the vaccine formulation contains 100 μg/mL pathogen antigen, 2-10 mg/mL of poly IC, 1-5 mg/mL of imiquimod, optional 1-5 mg/ml cetirizine and 45% PLGA of 50:50 lactide/glycolide, 7-17 kDa in N-methyl pyrrolidone or DMSO. The pathogen or tumor antigen can also be the antigen peptide that can bind with MHC to form MHC-peptide complex.

In one aspect, the current invention discloses composition and formulation to treat allergy comprising an antigen causing said condition and an immune activity enhancing agent in a sustained release formulation or implant. The antigen can be allergen, allergen or its fragment in form of its B cell antigen, T cell antigen in MHC-peptide complex form or the antigen peptide (or its derivative) of T cell antigen that can bind with MHC to form the MHC-peptide complex. Example of immune activity (or called immune function) enhancing agent can be selected from TLR agonist such as imiquimod, poly IC and CPG ODN. The current invention also discloses a method to treat allergy or inhibit IgE induced reaction by inducing antigen specific immune tolerance and/or inducing the production of competing IgG against the antigen in a subject by administering to the subject a said composition/formulation as either subcutaneous injection or intramuscular injections or intradermal injections or intralymphatic injection or an implant.

The current invention further discloses methods and regents to treat allergy by applying the combination/composition of antigen causing allergy and immune activity enhancing agent/drug either as a physical mixture or as synthetic conjugate or as nano/micro particles or liposome to the object/patient in need at a therapeutically effective amount. The combination/composition can be in a sustained (extended) release system such as an in-situ gelling system or implant.

Examples of suitable immune activity (function) enhancing agent include pattern recognition receptor (PRR) ligands, RIG-I-like receptor (RLR) ligands, Nod-like receptor (NLR) ligands, C-type lectin receptors (CLR) ligands, STING agonist, and Toll-like receptor ligands such as a TLR3 ligand, a TLR4 ligand, a TLR5 ligand, a TLR7/8 ligand, a TLR9 ligand, or a combination thereof. The immune function enhancing agent can be a vaccine adjuvant. Example of suitable vaccine adjuvant can be saponin such as Matrix-M adjuvant (quillaja saponins formulated with cholesterol and phospholipids into nanoparticles), squalene such as MF59 (an oil-in-water emulsion of squalene oil) and AS03 adjuvant (vitamin E and squalene oil-in-water emulsion), MPL such as AS01B, QS-21 which is purified from the bark of the quillaja saponaria, AS04 which is a combination of aluminum hydroxide and monophosphoryl lipid A (MPL), aluminum salts such as aluminum hydroxide, aluminum phosphate, alum (potassium aluminum sulfate), or mixed aluminum salts. The concentration of these vaccine adjuvants can be the same as currently used concentration or up to 20× higher. Preferably the Toll-like receptor ligand is a Toll-like receptors (TLR) agonist. Example includes imidazoquinoline family of TLR7/8 ligands (e.g. imiquimod(R837), gardiquimod, resiquimod (R848), 3M-052, 3M-852, 3M-S-34240, motolimod/VTX-2337, NKTR-262; CpG ODNs such as SD-101, ODN 1826 and ODN 2216, TLR agonist including TLR peptide agonist disclosed in patent applications WO2018055060A1, WO2013120073A1, WO2016146143A1 and US20180133295A1 and their citations, synthetic analogs of dsRNA, such as poly IC (e.g. poly ICLC, polyIC-kanamycin, polyI:polyC12U), TLR4/5 Ligands such as bacterial lipopolysaccharides (LPS, e.g. monophosphoryl lipid A), bacterial flagellin (e.g. vibrio vulnificus flagellin B), glucopyranosyl lipid A (GLA), TLR7 agonist Loxoribine or their derivatives/analogues, or their combinations. They can be in form of active drug, prodrug, liposome, emulsion, micelle, insoluble precipitate (e.g. in complex with condensing agent), conjugated to polymer drug carrier (e.g. dextran) or encapsulated in biodegradable microparticle/nanoparticle. Poly acrylic acid containing polymer such as Carbomer is also a immune function enhancing agent that can be used. Besides TLR agonist and STING agonist, other molecules that can activate/boost the function of immune system and immune cell such as APC, B cell and T cells can also be incorporated into the formulation.

Examples of suitable immune function activating/boosting molecule and additional suitable immune function enhancing agent can be found in US patent applications U.S. Ser. No. 15/945.741, U.S. Ser. No. 16/271,877 and U.S. Ser. No. 169/241,84 filed by the current inventor. They can be added to the formulation described here in at therapeutically effective amount to be used.

In some embodiments, the immune activity enhancing agent may comprise any of the agents provided herein. The immune activity enhancing agent can be a compound that directly provides the immune enhancing (e.g., activating) effect on APCs or it can be a compound that provides the immune enhancing effect indirectly (i.e., after being processed in some way after administration). Immune activity enhancing agents, therefore, include prodrug forms of any of the compounds provided herein. Different immune activity enhancing agent can be used as a mixture and be used in combination in the current invention. Immune activity enhancing agents also include nucleic acids that encode the peptides, polypeptides or proteins provided herein that result in an immune enhancing (e.g. activating) immune response. In embodiments, therefore, the immune activity enhancing agent is a nucleic acid that encodes a peptide, polypeptide or protein that results in an immune enhancing (e.g. activating) immune response. The nucleic acid can be coupled to synthetic nanocarrier. The nucleic acid may be DNA or RNA, such as mRNA. In embodiments, the inventive compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector.

Selecta's publications listed previously disclosed synthetic nanocarrier methods, and related compositions, comprising B cell and/or MEW Class II-restricted epitopes and immune activity suppressive agents in order to generate immune responses. In their disclosure, the antigen/epitope is conjugated to the nanocarrier and immune suppressive agent is coupled to the nanocarrier. An alternative method and composition are to use nano/micro particle having antigen/epitope causing allergy encapsulated within or non-covalently adsorbed to its surface and immune activity enhancing agent encapsulated within. The nano/micro particles can be made of biodegradable materials such as PLGA. These kinds of nano/micro particles (e.g. 10 nm˜10 μm of diameter in size) can be given to the patient in need as injection or inhaler or orally or applied topically to induce anti-allergy effect. The encapsulation of immune activity enhancing agent and antigen is well known to the skilled in the art and can be adopted from related publications readily. The surface of the nano/micro particles can have charged groups such as amino or carboxyl group to increase the binding of antigen/epitope causing allergy to its surface; it can also have a hydrophobic surface to allow binding antigen/epitope via hydrophobic interaction; or the combination of them. Introducing charged groups to the surface can be done by using surface modification or using amine or carboxyl group containing molecules to prepared the nano/micro particles. The antigen/epitope causing allergy can also be conjugated with a lipophilic moiety such as lipid molecule such as fatty acid or cholesterol to increase its binding to nano/micro particles. The adsorption of antigen/epitope causing allergy to the nano/micro particle surface can be done by incubating antigen/epitope with the nano/micro particle (e.g. 4° C. overnight in aqueous solution buffer such as 1×PBS) and then removing the unbound antigen/epitope (e.g. washing the nano/micro particle with aqueous buffer several times, similar to the ELISA plate coating procedure). In one example, 50 nm˜200 nm size PLGA nano particle encapsulated with 10% by weight of imiquimod prepared. Next the PLGA nano particle is mixed with allergen OVA (10 mg/mL) at 4C overnight to generate the OVA (ovalbumin) coated particle. The particle is washed 3 times with PBS to remove unbound OVA. In another example, imiquimod is dissolved in DMSO at 50 mg/ml. A total of 50 μL imiquimod is added to 1 ml PLGA (5 mg/ ml) dissolved in dichloromethane. Next the mixture is homogenized with 0.4 ml 5% OVA solution for 10 min using ultrasonication. The o/w emulsion is added to 2.1 ml of a 5% w/v solution of PVA to evaporate the organic solvent for 4 h at room temperature. OVA coated nano particles containing imiquimod are obtained after centrifugation at 3,500 g for 20 min. Additional washing step can be performed to obtain unbound OVA free particles. This OVA coated particle in an in-situ gelling system such as 2-5% alginate pH7 or 17-20% poloxamer 407 solution pH7 can be given to the target in need to induce OVA immune tolerance due to IgE to treat allergy against OVA as either subcutaneous injection or intramuscular injections or intradermal injections or intralymphatic injection. The OVA can be replaced with other allergen/epitope molecule to treat corresponding allergy. In another sample, lipophilic carboxylic acid or lipophilic amine or anionic detergent or cationic detergent (e.g. fatty acid such as caprylic acid, lauric acid; or cationic lipid such as DOTMA, DOTAP, cholesterylamine) can be added to the PLGA to prepare PLGA particle having surface charge. In one example, imiquimod is dissolved in DMSO at 50 mg/ml with lauric acid at 10 mg/mL. A total of 50 μL imiquimod/lauric acid is added to 1 ml PLGA (5 mg/ml PLGA) dissolved in dichloromethane. Next the mixture is homogenized with 0.1 ml 2% caprylic acid solution for 10 min using ultrasonication. The o/w emulsion is evaporated to remove the organic solvent for 4 h at room temperature. The resulting PLGA particle is washed 3 times with PBS and then incubated with OVA to prepare OVA bound particles. It can be added to an in situ gelling matrix and then be used to treat allergy to OVA. In one example, 1 mg˜10 mg of the particle in 3.5% sodium alginate and 1% HPMC can be injected to a patient with OVA intolerance every month for 3 times to induce OVA tolerance as subcutaneous injection or intralymphatic injection.

Furthermore, allergen (antigen)/epitope causing allergy can also be encapsulated within the nano/micro particle besides being conjugated or adsorbed to its surface. The preparation of antigen/epitope encapsulation is well known to the skilled in the art and can be adopted from related publications readily, e.g. using a double emulsion water/oil/water system.

Patent application US20130287729 disclosed antigen-specific, tolerance-inducing microparticles and uses thereof. It disclosed a microparticle (0.5 μm-10.0 μm in size) for targeting an antigen-presenting immune cell of interest and for inducing antigen-specific immune tolerance, wherein the microparticle comprises an antigen and a therapeutic agent wherein the therapeutic agent is an immunomodulatory agent, an immunosuppressive tolerogenic agent, or an agent that recruits the antigen-presenting immune cell of interest, wherein the surface of the microparticle comprises a ligand that targets the antigen-presenting immune cell of interest and the microparticle is made of biodegradable material. A further improvement of this method and composition to treat allergy is to use microparticle or nanoparticle having the size of 50 nm˜5μm, preferably made of biodegradable materials and use immune activity enhancing agent instead of the immunosuppressive agent preferably in a sustained release formulation such as in-situ gelling system or high viscosity formulation. The particle comprises an antigen causing allergy by encapsulation or coating or both. In some embodiments, the surface of the nano/micro particle is coated with Fc portion of an antibody or a full antibody with its Fc portion facing outside. This will bind with the FcR to facilitate APC uptake. In other embodiments, the surface of the nano/micro particle needs not to have a ligand that targets the antigen-presenting immune cell. In some embodiments, it can have antigen/epitope causing allergy coated on its surface. The inner part of the nano/micro particle contains immune activity enhancing agent listed in the current application and optionally antigen/epitope causing allergy, e.g. by encapsulation. The preparation method is well known to the skilled in the art and can be adopted from related publications readily. For example, 5 mg˜50 mg of the above particle containing gluten and poly IC in 3% sodium alginate with optional 0.5-2% HPMC, or in 15-25% Pluronic F127 or in 15-25% Poloxamer 407can be injected to a patient with gluten intolerance to induce gluten tolerance as subcutaneous or intramuscular or intralymphatic injection.

US patent application US20160338953 A1 disclosed a liposome-based immunotherapy. It provided a liposome encapsulating an autoantigen, wherein the liposome has a size comprised from 500 to 15000 nm and the liposome membrane comprises phosphatydilserine (PS) in an amount comprised from 10 to 40% by weight with respect to the total membrane liposomal composition. Pharmaceutical or veterinary compositions comprising a therapeutically effective amount of said liposome were also provided. Further, it provided liposomes and pharmaceutical or veterinary compositions as defined above for use as a medicament, particularly for the treatment of autoimmune diseases. The current invention also discloses antigen-specific liposome for allergy treatment and uses thereof. The liposome contains immune activity enhancing agent listed in the current application (and optionally antigen/epitope molecule causing allergy) inside by encapsulation. Optionally the surface of the liposome can also have allergy causing antigen/epitope coated. It can be given to the patient in need as injection to induce immune tolerance for allergen to treat allergy. The antigen/epitope causing allergy can also be conjugated with a lipid type molecule such as fatty acid or phospholipid or cholesterol derivative to allow it to be inserted to the liposome membrane. Suitable liposome can have a size between 50 nm˜20 μm. The preparation method and the protocol of its use are well known to the skilled in the art and can be adopted from related publications readily such as those in US20160338953. Example of the lipid molecule suitable for the current invention to prepare liposome includes but is not limited to phospholipid glycerolipid, glycerophospholipid, sphingolipid, ceramide, glycerophosphoethanolamine, sterol or steroid. These lipid molecules can also be used to prepare the allergy causing antigen/epitope-lipid conjugate. Membrane anchoring peptide-antigen/epitope conjugate can also be used instead of antigen/epitope-lipid conjugate. These liposome can be in a sustained release formulation such as in-situ gelling system or high viscosity formulation. For example, 5 mg˜50 mg of these liposome (5-25% of the formulation) containing egg white antigen such as ovomucoid and rapamycin in 3% sodium alginate with optional 0.5-2% HPMC, or in 15-25% Pluronic F127 or in 15-25% Poloxamer 407 can be injected to a patient with egg white intolerance monthly for 3 times to induce egg white tolerance as subcutaneous or intramuscular injection or intralymphatic injection at inguinal lymph node.

The current invention discloses methods, regents, compositions and formulations to treat allergy by injecting the mixture of antigen causing allergy and immune activity enhancing agent in a sustained release formulation such as in situ gelling system or implant to the object/patient in need. It can also contain anti-allergy drug such as antihistamines, corticosteroids, mast cell stabilizers, and leukotriene inhibitor. The addition of these anti-allergy drugs can prevent the allergy reaction induced by giving the allergen to the patient. The method and the said composition/formulation can be used to induce the generation of anti-allergen IgG antibody to compete the endogenous IgE which will generate allergy reaction; therefore, it will induce immune tolerance for the allergen. The immune activity enhancing agent can be in the form of active agent, prodrug form, microparticle or nanoparticle form or liposome form. The antigen causing allergy can be either B cell antigen/epitope or T cell antigen/epitope (e.g. MHC-peptide complex or conjugate; or the antigen fragment such as peptide that can bind with MHC) or their combination. The combination can be either B cell antigen/epitope with T cell antigen/epitope; or the combination of several different B cell antigen/epitope and/or several different T cell antigen/epitope targeting the same disease or different diseases. The use of peptide antigen (T cell epitope) that can bind with MHC to form MHC-peptide complex in vivo (T cell antigen) instead of the peptide-MHC complex reduce the size and molecular weight and can be used instead. The use of peptide antigen having single epitope domain can reduce the risk of activating mast cells by not cross linking the IgE on cell surface, therefore provide better safety yet still be cable to induce immune tolerance. The allergy causing antigen (allergen) used in the current invention can be either full allergen or its fragment such as its epitope, or their combination. Examples of them can be found in the current application and related publications and patent applications.

A mixture of allergy causing antigen and immune activity enhancing agent can be a physical mixture. A physical mixture means that the mixture of antigen and immune activity enhancing agent are simply mechanically mixed (e.g. by stirring or blending) together in their original form (e.g. liquid or solid form such as powder or particles) without any additional process (e.g. by mixing them in their original form together), or further size reducing process is applied before or after the mechanical mixing (e.g. crashing, grinding, mulling or homogenizing), or dispersed or dissolved separately in same or different type of liquid and then mix, or co-dispersed in liquid, or co-dissolved in solvent (e.g. water), and optional drying process (e.g. spray drying or lyophilization) can be applied with optional further size reducing process.

In some embodiments, the method is to use an in-situ gelling liquid containing both allergen or its fragment and immune enhancing drug (the drug listed above such as imiquimod or poly IC). It can also contain anti-allergy drug such as antihistamines, corticosteroids, mast cell stabilizers, and leukotriene inhibitor. Examples of the allergen can be pollen extract, dust mite extract, animal hair extract or food allergen such as nut/peanut/milk/egg protein. Alternatively, a biodegradable implant encapsulating allergy causing antigen and immune activity enhancing agent can be used for allergy treatment and prevention. The size of the implant can be bigger than 10 μm in diameter, preferably >100 μm, if the implant is a macro particle. The macro particle can be in the size range of 100 μm-10 mm. The particles can be made of biodegradable materials such as PLGA. The implant can also be non-sphere shape. For example, a 2 mm (length)×0.3 mm (diameter) rod made with PLGA containing 3 mg imiquimod and 0.5 mg gliadin or a 5 mm (length)×2 mm (diameter) rod made with PLGA containing 1 mg imiquimod and 5 mg gliadin can be used as an implant underneath the skin to treat gluten intolerance. Other implant format including non-degradable device can also be used such as NanoPortal Capsule™ from Nanoprecision Medical and Medici Drug Delivery System™ from Intarcia, as long as they can deliver the antigen and immune activity enhancing agent simultaneously. In some embodiments, sustained release implant such as NanoPortal Capsule™ and Medici Drug Delivery System™ can contain allergen only without the need of immune enhancing agent. Current allergen injection to treat allergy need to be injected very frequently, using the implant to provide sustained release of allergen will reduce the implantation (e.g. one implant every month or every 3 months) frequency and will be more patient friendly. The dose of the allergen in the first implant is low and increase gradually in the later implant to ensure the safety. The implant can also contain therapeutically effective amount (e.g. the dose currently used in clinic) of anti-allergy drug such as antihistamines, corticosteroids, mast cell stabilizers, and leukotriene inhibitor. In some embodiments, the implant can release a daily dose of antigen same as the dose of the current antigen injection used for allergy prevention/treatment and the release can last several weeks to several months. Preferably the released daily dose is able to induce tolerance to allergen but not cause severe allergy reaction such as severe immediate hypersensitivity reactions. For example, an implant contains 0.3-3 mg allergen which allows the release of 0.1 ug˜0.1 mg allergen daily for 30 days and the allergen can be peanut protein or egg white protein or pollen extract. The implant can be made of a material and in a configuration that allow it to be removed from the patient to increase its safety, if severe allergy reaction is observed, the implant. For example, it can be small cartridge that can be removed from patient which contains the allergen and optional TLR agonist or immunosuppressant in a sustained release system.

In one example, a composition and liquid formulation contains 0.5 ug˜100 m/mL gluten (e.g. G5004 gluten from wheat, Sigma) and 0.2 mg˜2/mg/mL of imiquimod or 0.2-2 mg/mL poly IC or their combination, in 2-5% sodium alginate pH=7 with optional 0.1% Tween-60 or optional 5% propylene glycol to improve drug solubility for gluten intolerance treatment as an injection. It can be lyophilized with optional bulking agent and reconstituted before use. Other allergen such as pollen extract, dust mite extract, animal hair extract or nut protein can be used instead and the concentration in the formulation can be the maximal tolerable concentration determined experimentally such as by subcutaneous injection of allergen.

In another example, a composition and liquid formulation contains 0.5-25 μg/ml gluten and 0.5-5 mg/ml of poly IC in 2-5% sodium alginate pH=7 with optional 2 mg/ml cetirizine. In another example, a composition and liquid formulation contains 5-50 μg/ml gluten, 0.5-2 mg/ml of STING agonist MK-1454 or 0.5-5 mg/ml of CpG ODN 1826 in 20% poloxamer 407. In another example, 1-100 μg/ml gluten and 0.1-1 mg/ml of imiquimod are mixed with in 1 mL 50% PLGA in N-methyl pyrrolidone or DMSO solution as the in-situ gelling formulation. In another example, a composition and liquid formulation contains 0.1 mg/ml gluten (e.g. G5004 gluten from wheat, Sigma) and 0.5 mg/ml of imiquimod or 20 μg/ml 3M-052 in 3.5% sodium alginate pH=7 and 1% HPMC. In another example, a composition and liquid formulation contains 5-500 μg/ml gluten (e.g. G5004 gluten from wheat, Sigma) and 1-5 mg/ml poly IC in in 19% poloxamer 407 and 0.5% hyaluronic acid. These formulations can be used to induce gluten tolerance and treat gluten intolerance as either subcutaneous injection or intramuscular injections or intradermal injections or intralymphatic injection. The gluten can be replaced with gliadin such as deamidated gliadin instead. In some embodiments, the gluten or deamidated gliadin peptide containing formulation can be injected to the patients at their maximal tolerable dose. The gluten in the above examples can be replaced with egg white protein such as 2-100 μg/ml of ovomucoid (Gal d 1) or 5-100μg/ml ovalbumin (Gal d 2) or their combination with optional 2-100 μg/ml ovotransferrin (Gal d 3) and 2-100 μg/ml lysozyme (Gal d 4) to treat egg white allergy. In another example, a composition and liquid formulation contains peanut antigen ara h2 0.01-1 μg/ml and 1 mg/ml in 3.5% sodium alginate pH7 to treat peanut allergy. In one example, a composition and liquid formulation contains peanut antigen ara h2 0.5 μg/ml, 0.5 mg/ml of imiquimod in 20% Poloxamer 407 as injection to treat peanut allergy. In one example, a composition and liquid formulation contains peanut antigen ara h2 0.2 μg/ml, 0.2 mg/ml imiquimod in 45% 50:50 lactide/glycolide in N-methyl pyrrolidone or DMSO to treat peanut allergy. The immune activity enhancing agent drug or both the immune activity enhancing agent drug and the allergy causing antigen can be either in the form of powder or gel or semi liquid or in the form of liposome (e.g. 100 nm˜5 μm diameter) or in a nano/micro particle (e.g. 100 nm˜1 μm) or being conjugated to a dendrimer or linear polymer (e.g. couple to poly acrylic acid or poly sialic acid via ester bond to form a polymer based prodrug with MW=5K˜500K Dalton) and then formulated in the in situ gelling system or high viscosity liquid. Other pharmaceutically acceptable amount of antigen causing allergy and immune activity enhancing agent can also be used, as long as it can produce satisfactory biological and therapeutical (e.g. immune tolerance) effect, which can be determined experimentally by screening and testing with well-known protocol and methods.

Other allergen such as pollen extract, dust mite extract, animal hair extract and food allergen such as nut/peanut/milk/egg extract can also be used instead in the above formulations to treat related allergy. Example of the concentration of these allergens can be between 0.1 μg/ml to 1 mg/mL as long as they are tolerable by the subject in need. During the treatment, the concentration of the allergen in the formulation can increase when the patient's tolerance increases, which is the standard practice of the allergy immune therapy.

The amount of allergen, which corresponds to a given level of potency, varies strongly depending on the allergen specie. In a further embodiment of the invention the concentration of major allergen in a mono-dose is can be from 0.05 to 500 μg, such as from 0.1 μg to 100 μg in the injection. In the field of allergy extracts, there is no international accepted standardization method. A number of different units of extract strength i.e. bio-potency exist. The methods employed and the units used normally measure the allergen content and biological activity. Examples hereof are SQ-Units (standardized quality units), BAU (biological allergen Units), BU (biological units), UM (units of mass), IU (international units) and IR (index of reactivity). Hence, if extracts of origins other than those disclosed herein are used, they need to be standardized against extract disclosed herein in order to determine their potency in SQ units or any of the above mentioned units. The bio-potency, i.e. the in vivo allergenic activity, of a given extract depends on a number of factors, the most important being the content of major allergens in the extract, which varies with the composition of the biological source material. The amount of allergen extract in grams to be used for obtaining a desired bio-potency varies with the type of extract in question, and for a given type of extract the amount of allergen extract varies from one batch to another with the actual bio-potency of the extract. For a given batch of extract, the amount of allergen extract in grams to be used for obtaining a desired bio-potency may be determined using the procedure described in US patent number U.S. Pat. No. 9,248,097B2.

The SQ-Unit is determined in accordance with SQ biopotency standardization method, where 100,000 SQ units equal the standard subcutaneous maintenance dose. Normally 1 mg of extract contains between 100,000 and 1,000,000 SQ-Units, depending on the allergen source from which they originate and the manufacturing process used. The precise allergen amount can be determined by means of immunoassay i.e. total major allergen content and total allergen activity. BAU is biological potency units as determined according to the requirements of the FDA for allergen product. A dose of 100,000 SQ-Units containing grass extract equals a content of 2600-4700 BAU according to the method above. Likewise, other extracts can be assessed according to the method above.

Current invention also discloses methods and regents to treat or prevent allergy by applying the mixture of allergy causing antigen and said immune activity enhancing agent/drug as injection to the object/patient in need. The injection can be given as either subcutaneous injection or intramuscular injections or intradermal injections. The injection can contain a viscosity enhancing agent to increase its viscosity when it is being injected, which acts as a sustained release formulation of both antigen and immune activity enhancing agent. Allergy causing antigen and immune activity enhancing agent can be either in free molecule form or in nano/micro particle from including liposome form. In certain embodiments, the injection has a viscosity greater than 5,000 cps at room temperature. In certain embodiments, the injection has a viscosity greater than 50,000 cps at room temperature. In certain embodiments, the injection has a viscosity greater than 500,000 cps at room temperature. In certain embodiments, the injection has a viscosity of 1,000,000 cps at room temperature. Example of the viscosity enhancing agent can be found readily from known pharmaceutical acceptable excipient such as hyaluronic acid (linear or crosslinked), starch and carbomer. In some embodiments, the viscosity enhancing agent is biodegradable. In one example, a viscous injection contains 0.1-500 ug/mL gluten (e.g. G5004 gluten from wheat, Sigma) and 0.1-0.5 mg/mL of imiquimod or 100 ug-0.5 mg/mL poly IC and suitable amount of hyaluronic acid (e.g. 10-50 mg/mL linear or cross linked hyaluronic acid) to reach a viscosity of 300,000 cps. In one example, a viscous injection contains 0.1-10 ug/mL gluten (e.g. G5004 gluten from wheat, Sigma) and 50 ug mg/mL of imiquimod or 100 μg/mL poly IC and suitable amount of hyaluronic acid (e.g. 20-50 mg/mL cross linked hyaluronic acid) to reach a viscosity of 500,000 cps with optional 2 mg/ml cetirizine.

The injection formulation can also be a thermal phase changing formulation as those described previously. A thermal phase changing injectable formulation containing both antigen and immune activity enhancing agent can be given as either subcutaneous injection or intramuscular injections or intradermal injections to induce antigen specific immune tolerance and treat corresponding autoimmune diseases or allergy. For example, a composition of a thermal phase changing injectable formulation is 0.01-0.1 mg/mL gluten (e.g. G5004 gluten from wheat, Sigma) and 0.2-1 mg/mL imiquimod in 25% (w/w) Poloxamer-407 pH=7 solution and optional 2 mg/ml cetirizine, which can be injected to a patient with gluten intolerance 0.1-1 mL to treat allergy to gluten as subcutaneous injection or intralymphatic injection. The gluten in the above examples can be replaced with egg white protein such as 5-100 μg/ml of ovomucoid (Gal d 1) or 5-100 μg/ml ovalbumin (Gal d 2) or their combination with optional 5-100 μg/ml ovotransferrin (Gal d 3) and 5-100 μg/ml lysozyme (Gal d 4) to treat egg allergy.

The formulations including implant to treat allergy in the current invention can also contain anti-allergy drug as described previously. The amount of anti-allergy drug added can be the same as those currently used in anti-allergy treatment. The addition of these anti-allergy drugs can prevent the allergy reaction induced by giving the allergen to the patient. Furthermore, those reagents and formulations can also be injected into lymph node instead for allergy treatment. Intralymphatic allergen administration is known and the same procedure can be readily adopted for the current invention. In some embodiments, the amount of the reagent or formulation injected into lymph node is between 0.1 ug˜0.1 mg allergen with injection volume between 0.1 ml to 1 ml per lymph node monthly for 3 months.

The immune activity enhancing agent can also be conjugated to carbohydrate polymer or other bio compatible polymer (e.g. dextran or heparin or hyaluronic acid or poly peptide) to form prodrug as described in U.S. patent application Ser. No. 15/723,173; Ser. No. 16/380,951 and Ser. No. 16/029,594. The novel prodrugs can be in the form of carbohydrate (or other polymer) drug conjugate in which the drug can be conjugated to the carbohydrate (or other polymer) with cleavable linkage. More than one drugs can be conjugated to the polymer backbone. Suitable carbohydrate includes sialic acid containing polymer, hyaluronic acid, chondroitin sulfate, dextran, carboxyl dextran, cellulose, carboxyl cellulose and their derivatives. It can also be a linear polymer backbone (e.g. dextran or synthetic polymer such as PVA, PAA). Furthermore, the immune enhancing drug can also be directly conjugated to antigen or conjugated to the antigen via a linker or carrier. The carrier can be a polymer. The allergy causing antigen can be conjugated to a carrier to form a multimer. The allergy causing antigen and immune enhancing drug can also be conjugated together. They can be in the form of linear polymer, micro particle, nano particle, liposome or implant. A carrier system can be used for the previous and current applications to construct the conjugate. For example, the liposome or microparticle or nanoparticle can be used as a carrier. The antigen can be immobilized on the surface of the liposome or particles and the immune enhancing agent can be either encapsulated inside or co-immobilized on the surface of liposome or particles. The carrier can also be a linear or branched polymer such as dextran, hyaluronic acid, heparin, chondroitin sulfate and poly peptide. Both allergy causing antigen and immune enhancing agent can be conjugated to the polymer. They can be given to the subject in need to treat allergy by administering to the subject said conjugate (e.g. subcutaneous injection). Additional details can be found in the previous disclosures.

When liposome is used, either the immune enhancing drug or both the antigen+drug can be encapsulated in the liposome. The injectable formulation or implant can contain either antigen+drug or antigen-drug conjugate or encapsulated antigen/drug (e.g. in microsphere or liposome) or their combinations. The antigen can be either in the form of crude antigen (e.g. peanut extract, gluten, pollen extract, dust mite extract) or purified antigen (e.g. peanut antigen protein ara h2, gliadin) or antigen-drug conjugate or encapsulated antigen (e.g. in microsphere or liposome) or their mixture.

Another format suitable for the current application is to use microsphere in the sustained release formulation such as in-situ gelling system. The term microsphere includes particles from nano meter size to micrometers (e.g. 50 nm˜50 μm in diameter). Preferably the microsphere is bio degradable (e.g. made of biodegradable polymer such as poly(lactidecoglycolide), PLGA), the microsphere can further encapsulate immune suppressive drug such as imiquimod (e.g. 1%˜80% weight of the microsphere). For example, the microsphere can be biodegradable synthetic polymer such as PLGA. Immune enhancing drug such as imiquimod (e.g. 1%˜80% weight of the microsphere) is encapsulated. The size of the microsphere is 3 μm or 300 nm. Antigen is also conjugated to the surface of the microsphere directly or with a linker. The antigen can also be encapsulated in the microsphere as well. Alternatively, the drug (immune activity enhancing agent) can be conjugated to the surface of the microsphere instead of being encapsulated. Examples of microsphere suitable for the current application can be readily adopted from the disclosure in the publications such as those in patent application number U.S. Ser. No. 13/880,778, U.S. Ser. No. 14/934,135, CA 2910579, U.S. Ser. No. 13/084,662 and US patent U.S. Pat. No. 8,652,487 and other patent application filed by Selecta Biosciences. It can be formulated as an in-situ gelling formulation or high viscosity liquid to be used to treat allergy, which can be either injected or implanted (the formulation is encapsulated inside the implant) to the patient.

Another format suitable for the current application is to use polymer carrier conjugated with allergen and immune activity enhancing agent in sustained release formulation such as said in- situ gelling matrix. The polymer is conjugated with multiple antigen (e.g.1-100), and multiple copies of immune activity enhancing agent (e.g. 5˜500 molecules).

The formulation/composition of the current invention can contain increased dose of allergen in later stage similar to the dosing protocol used by current treatment protocol using allergen (oral or topical or injection). That is, the treatment involves a series of formulations, the first formulation contains lowest amount of allergen that can be tolerated and it gradually increases over time in the later formulation while the amount of other drug if present (e.g. immune enhancing agent or immunosuppressant) can be unchanged. The allergen amount in the first formulation can be the highest amount of allergen that can be tolerated by patient without causing severe allergenic reaction. In one example, the patient began with a first single dose of injectable formulation containing 0.1 ug of egg white protein either in PBS, after the initial dose, subject received approximately doubling doses of egg white protein extract every 30 minutes until the highest tolerated single dose is determined. Based on the highest tolerated single dose, subject begins weekly dosing with the formulation containing highest tolerated dose of egg white protein and 0.2 mg imiquimod in 0.5 mL 3.5% sodium alginate, 1 dose weekly for 2 weeks. As long as subject is tolerating current doses, the egg white in the formulation containing 0.2 mg imiquimod in 0.5 mL 3.5% sodium alginate are increased as the table every 2 weeks until reaching 200 ug. Once subjects reached the dose of 200 ug, they are instructed to take this dose every 6 month for 2 years. Similarly, the imiquimod can be replaced with 0.1-0.5 mg rapamycin or methotrexate instead. Alternatively, no TLR agonist and no immunosuppressant is used in the sustained release formulations containing antigen. Other allergen such as pollen extract, dust mite extract, animal hair extract and food allergen such as nut/peanut/milk/egg extract can also be used instead in the above formulations to treat related allergy. Example of the concentration of these allergens can be between 0.1 μg/ml to 1 mg/mL depend on the species and patient tolerance as long as they are tolerable by the subject in need. During the treatment, the concentration of the allergen in the formulation can increase when the patient's tolerance increases, which is the standard practice of the allergy immune therapy.

These formulations and compositions containing allergen and adjuvant type agent (e.g. TLR agonist or STING agonist) is mainly used to treat allergy by producing allergen specific IgG competing endogenous IgE which cause allergy reaction. They are not intended to treat autoimmune disease against self-antigen and not for the prevention of anti-drug antibody. Preferably the adjuvant type agent is a Th1 biasing immunostimulatory agent, comprises STING agonist, natural or synthetic agonist (for example TLR-1, TLR-2, TLR-3, TLR-4, TLR-5, TLR-6, TLR-7, TLR-8, TLR-9, TLR-10 and TLR-11 agonist) for Toll-like receptor (TLR) including immidazoquinolinaminas such as 2-bridge joint immidazoquinolinaminas, imidazopyridine amine, cycloalkyl imidazopyridine amine, CpG, immunostimulating RNA, lipopolysaccharide, VSV-G or the HMGB-1 such as imiquimod, R848, 3M-052 and poly IC. In some preferred embodiments, the TLR agonist used is selected from imiquimod and poly IC.

In one aspect, the current invention discloses compositions and formulations comprising one or more antigen causing allergy and optional vaccine adjuvant type agent (e.g. TLR agonist, STING agonist) in a sustained (extended) release system such as an in-situ gelling system or implant or high viscosity liquid to treat allergy. The current invention also discloses a method to treat allergy in a subject by administering to the subject these compositions and formulations as an injection or implant. In some embodiments, said formulations is prepared by mixing a ready to use allergen containing product used to desensitize patient (e.g. those clinically used allergen extract product such as those subcutaneous allergy immunotherapy (SCIT) injections from Alk Abello AS with a sustained release system matrix on site before use and then the mixture is used as a final formulation to be injected to the patient. Said sustained release system matrix is a composition/formulation containing suitable amount of sustained release system material such as an in-situ gelling material or high viscosity liquid and optional adjuvant type agent (e.g. TLR agonist, STING agonist). The concentration of the in-situ gelling material in said sustained release system matrix need to be high enough to provide desired gelling effect after being mixed with the allergen extract product, which can be determined based on the dilution factor caused by the addition of allergen extract product. Preferably after mixing together the final formulation for injection has a pH value between 6-8 and the osmolarity is close to physiological osmolarity, which can be controlled by adjusting the pH buffer capacity, pH value, osmolarity of the sustained release system matrix based on the mixing ratio. In some embodiments, the composition/formulation is a liquid containing optional adjuvant type agent and suitable amount of self-gelling polymers disclosed in the current invention or its lyophilized form with optional bulking agent/lyoprotectant added before lyophilization, the amount of said polymer need to be enough to form gel in vivo after it is mixed with the allergen containing product used to desensitize patient. In some embodiments, the composition/formulation is a 2˜50% sodium alginate in water or saline and the pH is 5-8 by the addition of concentrated base or acid such as NaOH or HCl. In some embodiments, the composition and the formulation is 2˜20% sodium alginate in water or saline and the pH is 7-8 with the addition of 2M NaOH or 2M HCl. In some embodiments the formulation's osmolality is adjusted with physiological acceptable excipient to have an osmolality similar to physiological condition. In some embodiments the formulation has low osmolality and low pH buffering capacity so it will not affect the osmolality and pH value of the allergen containing formulation after being mixed together especially for those solid type (e.g. lyophilized form) preformulated allergen containing drug product; for example, the formulation has osmolality and pH buffering capacity lower than 0.5× PBS. Calcium salt or other divalent cationic salt can be incorporated in the formulation at a low concertation that will not cause gelling in vitro. For example, 2-20 mg of Ca2+ per 1 g of alginate can be used. The low contraction of Ca2+ salt will help the gelling in vivo, e.g. 0.05%˜0.5% calcium gluconate in the formulation. The formulation can further comprise gelling enhancing polymers as previously disclosed such as 0.1˜1% HA, CMC, HPMC, carbomer, MC, chitosan; 10-30% poloxamer or their combinations. Preferably the final solution will have 2˜6% alginate after mixing. In some embodiments, therapeutically effective amount of TLR agonist (e.g. poly IC or imiquimod or R848 or 3M-052 or CPG ODN such as CPG ODN 1018 or their combinations) or STING agonist or their combinations can be further incorporated into the said alginate containing matrix formulation. In one example, the matrix formulation is 5% sodium alginate, optional 1% HPMC and optional 0.2-2 mg/mL poly IC and optional 0.2-2 mg/mL CPG ODN 1018 in 1× PBS having a pH value of 7; 1 ml of it is mixed with 1 mL Alutard SQ dust mite extract (1-10 Allergy Unit/mL dilution) and the resulting final formulation can be injected to the patient to treat dust mite allergy. Other allergen extract such as cat hair allergen extract, pollen allergen extract, mixed vespid venom protein can also be used instead of dust mite extract to treat related allergy. For example, the matrix formulation is a saline solution containing 6% sodium alginate and optional 0.2-2 mg/mL imiquimod and optional 5 mg/mL diphenhydramine HCl as anti-histamine agent in 0.5% NaCl with a pH value at 7 to be mixed with a suitable strength of bermuda grass pollen extract at 1:1 ratio to generate a final formulation to be injected to the patient as subcutaneous or intramuscular injection or intralymphatic injection or being injected proximal to the lymph node at 0.2-0.5 ml volume. In some embodiments, the alginate can be replaced with 3-6% hyaluronic acid to form a viscous solution. In one example, the matrix formulation is 5% hyaluronic acid and optional 5 mg/mL poly IC in water having a pH value of 7; 1 ml of it is mixed with 0.5 mL Alutard SQ dust mite extract (1-10 Allergy Unit/mL dilution) and the resulting final formulation can be injected to the patient to treat dust mite allergy. In some embodiments, the alginate can be replaced with 30-50% Pluronic F127 or poloxamer 407 in the matrix formulation and after mixing the final concentration of Pluronic F127 or poloxamer 407 should be greater than its in situ gelling concentration (e.g. >17%). In one example, the matrix formulation is 30% poloxamer 407 and optional 0.2-2 mg/mL poly IC in water having a pH value of 7; 1 ml of it is mixed with 0.5 mL Alutard SQ dust mite extract (1-10 Allergy Unit/mL dilution) and the resulting final formulation can be injected to the patient to treat dust mite allergy. In some embodiments, PLGA organic solvent solution can be used as in situ gelling agent in the matrix formulation and after mixing the final formulation should not cause gelling in vitro but still form gel in vivo, which can be controlled by adjusting the ratio of PLGA solution and allergen extract product and the concentration of PLGA. In one example, the matrix formulation is 60% 50:50 lactide/glycolide PLGA in in N-methyl pyrrolidone or DMSO and optional 0.1-1 mg/mL imiquimod and optional 5 mg/mL diphenhydramine. 1 ml of it is mixed with 0.1 mL Alutard SQ dust mite extract (10-50 Allergy Unit/mL dilution) and the resulting final formulation can be injected to the patient to treat dust mite allergy. Alternatively, the adjuvant like agent in above matrix formulations can be replaced with therapeutically effective amount of immunosuppressant described previously such as mTOR inhibitor (e.g. 0.1-2 mg/mL rapamycin or its analogue or methotrexate) to treat allergy. In one example, the matrix formulation is 5% sodium alginate, optional 1% HPMC and 0.2 mg/mL rapamycin and optional 0.1% tween-20 as solubility enhancer in 1× PBS having a pH value of 7; 1 ml of it is mixed with 1 mL Alutard SQ dust mite extract (1-10 Allergy Unit/mL dilution) and the resulting final formulation can be injected to the patient to treat dust mite allergy. Other allergen extract such as cat hair allergen extract, pollen allergen extract, mixed vespid venom protein can also be used instead of dust mite extract to treat related allergy. For example, the matrix formulation is a saline solution containing 6% sodium alginate and optional 0.1-1 mg/mL rapamycin and optional 5 mg/mL diphenhydramine HCl as anti-histamine agent in 0.5% NaCl with a pH value at 7 to be mixed with a suitable strength of bermuda grass pollen extract at 1:1 ratio to generate a final formulation to be injected to the patient as subcutaneous or intramuscular injection or intralymphatic injection or being injected proximal to the lymph node at 0.2-0.5 ml volume. In one example, the matrix formulation is 3% crosslinked sodium hyaluronic acid and optional 1 mg/mL rapamycin in water having a pH value of 7; 1 ml of it is mixed with 0.5 mL Alutard SQ dust mite extract (1-10 Allergy Unit/mL dilution) and the resulting final formulation can be injected to the patient to treat dust mite allergy. In some embodiments, the alginate can be replaced with 30-50% Pluronic F127 or poloxamer 407 in the matrix formulation and after mixing the final concentration of Pluronic F127 or poloxamer 407 should be greater than its in situ gelling concentration (e.g. >17%). In one example, the matrix formulation is 30% poloxamer 407 and optional 0.3 mg/mL rapamycin or 0.3 mg/mL methotrexate in water having a pH value of 7; 1 ml of it is mixed with 0.5 mL Alutard SQ dust mite extract (1-10 Allergy Unit/mL dilution) and the resulting final formulation can be injected to the patient to treat dust mite allergy. In some embodiments, PLGA organic solvent solution can be used as in situ gelling agent in the matrix formulation and after mixing the final formulation should not cause gelling in vitro but still form gel in vivo, which can be controlled by adjusting the ratio of PLGA solution and allergen extract product and the concentration of PLGA. In one example, the matrix formulation is 60% 50:50 lactide/glycolide PLGA in in N-methyl pyrrolidone or DMSO and 0.1-1 mg/mL rapamycin and optional 5 mg/mL diphenhydramine. 1 ml of it is mixed with 0.1 mL Alutard SQ dust mite extract (10-50 Allergy Unit/mL dilution) and the resulting final formulation can be injected to the patient to treat dust mite allergy. Therefore, in one aspect, the current invention discloses compositions and formulations comprising one or more antigen causing allergy and an immunosuppressant in a sustained (extended) release system such as an in-situ gelling system or implant to treat allergy. The current invention also discloses a method to treat allergy in a subject by administering to the subject a said compositions and formulations as an injection. Said formulations is prepared by mixing a ready to use allergen containing product used to desensitize patient (e.g. those clinically used allergen extract product) with a sustained release system matrix containing immunosuppressant on site before use and then used as a final formulation to inject to the patient. Said sustained release system matrix is a composition/formulation containing suitable amount of sustained release system material and immunosuppressant as an in-situ gelling material or high viscosity liquid.

Another two type of ready to use product in allergy immunotherapy (AIT) treatments are sublingual drop or tablet. Sublingual allergy immunotherapy (SLIT) liquid drops are administered under the tongue. Patients administer the drops themselves at home, avoiding the need for regular visits to the doctor. Tablets are administered by the patient at home and are often sublingual tablet placed under the tongue. Tablets for house dust mite, grass, ragweed and Japanese cedar allergies are already available in many markets. Said sustained release system matrix above can also be used in combination with the sublingual drops or tablet by placing both sustained release system matrix and drops or tablet under the tongue. As described above, said sustained release system matrix can contain either immunosuppressant such as rapamycin or methotrexate, or adjuvant type agent such as imiquimod or poly IC, or no active drug at all. It can be either said in situ gelling formulation or said high viscosity liquid. For example, the matrix formulation is a saline solution containing 2% sodium alginate and optional 1 mg/mL rapamycin and optional 5 mg/mL diphenhydramine HCl as anti-histamine agent in 0.5% NaCl with a pH value at 7, a patient can add one or 2 drop of this matrix and the commercial SLIT drop or tablet under the tongue at the same time to treat allergy or mix them together then place the mixture under tongue. In another example, 2% sodium alginate is replaced with 1% HPMC so the matrix is a high viscosity solution instead. In another, the matrix formulation is a saline solution containing 2% sodium alginate and optional 1 mg/mL poly IC or 0.5 mg/mL imiquimod and optional 5 mg/mL diphenhydramine HCl as anti-histamine agent in 0.5% NaCl with a pH value at 7, a patient can add one or 2 drop of this matrix and the commercial SLIT drop or tablet under the tongue daily or weekly to treat allergy. In some embodiments, the matrix is not a sustained delivery system so viscosity enhancing agent or gelling agent in the matrix is not required. The matrix is just a solution containing effective amount of either immunosuppressant such as rapamycin or methotrexate, or adjuvant type agent such as imiquimod or poly IC. In some embodiments, the concentration of rapamycin or methotrexate or imiquimod or poly IC is between 0.1-10 mg/mL. Optional transdermal enhancing agent and solubility enhancing agent such as tween-80, DMSO, transcutol can also be incorporated in the solution. In one example, the solution is 1 mg/mL rapamycin in 1× PBS with 0.1% tween-80. A patient can add one or 2 drop of this solution and the commercial SLIT drop or tablet under the tongue to treat allergy. In another example, the solution is 1 mg/mL polyIC in 1× PBS. A patient can add one or 2 drop of this solution and the commercial SLIT drop or tablet under the tongue daily to weekly to treat related allergy.

In some embodiments, a tablet or membrane containing effective amount of either immunosuppressant such as rapamycin or methotrexate, or adjuvant type agent such as imiquimod or poly IC is used instead of the liquid matrix described above. Said tablet or membrane can be used in combination with the SLIT drops or SLIT tablet by placing both said tablet/membrane and the SLIT drops or SLIT tablet under the tongue or in the mouth to treat related allergy. In some embodiments, the concentration of rapamycin or methotrexate or imiquimod or poly IC is between 0.05-0.5 mg per tablet/membrane. The tablet/membrane is essentially a sublingual dissolvable tablet/membrane. Optional transdermal enhancing agent such as tween-80, DMSO, transcutol can also be incorporated in the tablet or membrane. In one example, a 5 mm diameter tablet contains 0.25 mg rapamycin or 1 mg poly IC or 0.2 mg imiquimod or 0.5 mg methotrexate, 40% mannitol, 40% lactose, 1% sodium cyclamate, 1% PVP K30 and 1% magnesium stearate. A patient can add one such tablet and the commercial SLIT drop/tablet under the tongue close to each other at the same time to treat allergy daily to weekly. In one example, a 3 mm diameter tablet contains 0.1 mg rapamycin or 0.5 mg poly IC, 2 mg anti-histamine drug, 20% sucrose, 50% lactose, 25% polyethylene glycol 6000 and 1% PVP K30. A patient can add one such tablet and a commercial SLIT drop/tablet under the tongue close to each other at the same site daily to weekly to treat allergy. The tablet can also be used with other orally used allergy immunotherapy medicine such as those described in patent number U.S. Pat. No. 9,271,899B2 and CN103025303A. Alternatively, the allergen and the immunosuppressant, or allergen and adjuvant type agent, can also be combined in one tablet and used for SLIT treatment. These tablets can be essentially the same SLIT tablet currently used with additional immunosuppressant or additional adjuvant type agent incorporated within the tablet. For example, a SLIT tablet has the same or similar composition as Oralair tablet (Stallergenes) except containing additional 0.1-0.2 mg rapamycin or 0.1-0.2 mg methotrexate to treat symptoms of allergies to the grass pollens. In another example, a SLIT tablet has the same or similar composition as Acarizax/Odactra tablet except containing additional 0.1-0.2 mg rapamycin or 0.1-0.2 mg methotrexate and 1 mg cetirizine or 1 mg corticosteroid such as fluticasone or budesonide to treat symptoms of allergies to the dust mite. In another example, a SLIT tablet has the same or similar composition as Acarizax/Odactra tablet except containing additional 0.1-0.2 mg imiquimod or 0.1-0.2 poly IC and 1 mg diphenhydramine HCl to treat symptoms of allergies to the dust mite. When the allergen containing drug is intended to be taken orally in to digestive system instead sublingually such as the PALFORZIA peanut allergen powder from Aimmune Therapeutics, additional immunosuppressant or additional adjuvant type agent can be incorporated within the tablet or capsule. For example, an enteric coated capsule or tablet containing 1-100 mg defatted peanut flour and 0.1-1 mg rapamycin or 0.1-1 mg methotrexate or 100-500 mg sialic acid or polysialic acid can be taken orally to treat peanut allergy.

In one aspect, the current invention discloses compositions and formulations comprising a drug that can produce anti-drug antibody and an immunosuppressant in a sustained (extended) release system such as an in-situ gelling system or implant or high viscosity liquid such as those described previously to treat anti-drug antibody. The current invention also discloses a method to treat or inhibit anti-drug antibody production in a subject by administering to the subject a said compositions and formulations as an injection. In some embodiments, said formulations is prepared by mixing a drug that produce ADA (anti-drug antibody), e.g. those clinically used protein drugs including antibody, with a sustained release system matrix on site before use and then the mixture is used as a final formulation as inject to the patient. Said sustained release system matrix is a composition/ formulation containing suitable amount of sustained release system material as an in-situ gelling material or high viscosity liquid and therapeutically effective amount of immunosuppressant described previously such as mTOR inhibitor (e.g. 0.1-5 mg/mL rapamycin or methotrexate).

The concentration of the in-situ gelling material in said sustained release system matrix need to be high enough to provide desired gelling effect after being mixed with drug product that generate ADA, which can be determined based on the dilution factor caused by the addition of drug product that generate ADA. Preferably after mixing together the final formulation for injection has a pH value between 6-8 and the osmolarity is close to physiological osmolarity, which can be controlled by adjusting the pH buffer capacity, pH value, osmolarity of the sustained release system matrix based on the mixing ratio. In some embodiments, the composition/formulation is a liquid containing immunosuppressant and suitable amount of self-gelling polymers disclosed in the current invention or its lyophilized form with optional bulking agent/lyoprotectant added before lyophilization, the amount of said polymer need to be enough to form gel in vivo after it is mixed with the drug that generate ADA. In some embodiments, the composition/formulation is a 2˜20% sodium alginate and 0.1-2 mg/mL rapamycin in water or saline and the pH is 5-8 by the addition of concentrated base or acid such as NaOH or HC1. In some embodiments, the composition and the formulation is 2˜20% sodium alginate and 1-5 mg/mL rapamycin in water or saline and the pH is 7-8 with the addition of 2M NaOH or 2M HCl. In some embodiments the formulation's osmolality is adjusted with physiological acceptable excipient to have an osmolality similar to physiological condition. In some embodiments the formulation has low osmolality and low pH buffering capacity so it will not affect the osmolality and pH value of the ADA generating drug containing formulation after being mixed together especially for those solid type (e.g. lyophilized form) preformulated drug product that can generate ADA; for example, the formulation has osmolality and pH buffering capacity lower than 0.5× PBS. Calcium salt or other divalent cationic salt can be incorporated in the formulation at a low concertation that will not cause gelling in vitro. For example, 2-20 mg of Ca2+ per 1 g of alginate can be used. The low contraction of Ca2+ salt will help the gelling in vivo, e.g. 0.05%˜0.5% calcium gluconate in the formulation. The formulation can further comprise gelling enhancing polymers as previously disclosed such as 0.1˜1% HA, CMC, HPMC, carbomer, MC, chitosan; 10-30% poloxamer or their combinations. Preferably the final solution will have 2˜6% alginate after mixing. In some embodiments, therapeutically effective amount of immunosuppressant other than mTOR inhibitor, e.g. IL-2, TGF-β, PD-L1, IL-15, IFN-y, IL-10, IL-21, IL-27, IL-2/anti-IL-2 antibody complexes or their mimics or derivatives such as a pegylated IL-2 NKTR-358 or their combinations, can be further incorporated into the said alginate containing matrix formulation. In one example, the matrix formulation is 5% sodium alginate, optional 1% HPMC and 0.2 mg/mL rapamycin and optional 200-2000 IU/mL IL-2/anti-IL-2 antibody complexes in 1× PBS having a pH value of 7; 1 ml of it is mixed with 1mL Humira (20 mg/0.4 mL drug product) and the resulting final formulation can be injected to the patient to treat ADA against Humira or prevent ADA against Humira. Other drugs such as other recombinant proteins such as antibody or growth hormone or virus vector be used instead of Humira to treat or prevent related ADA. For example, the matrix formulation is a saline solution containing 6% sodium alginate and 0.2 mg/mL rapamycin and optional 0.1% Tween-20 in 0.5% NaCl with a pH value at 7 to be mixed with a drug product containing suitable amount of AAV virus (e.g. 10\hat{ }10 copy/mL) at 1:1 ratio to generate a final formulation to be injected to the patient as subcutaneous or intramuscular injection or intralymphatic injection or being injected proximal to the lymph node at 0.2-0.5 ml volume to prevent T and B cell immunity against AAV. In some embodiments, the alginate can be replaced with 30-50% Pluronic F127 or poloxamer 407 in the matrix formulation and after mixing the final concentration of Pluronic F127 or poloxamer 407 should be greater than its in situ gelling concentration (e.g. >17%). In one example, the matrix formulation is 30% poloxamer 407 and 0.5 mg/mL rapamycin in water having a pH value of 7; 1 ml of it is mixed with 0.5 mL Humira and the resulting final formulation can be injected to the patient to treat or prevent Humira ADA. In some embodiments, PLGA organic solvent solution can be used as in situ gelling agent in the matrix formulation and after mixing the final formulation should not cause gelling in vitro but still form gel in vivo, which can be controlled by adjusting the ratio of PLGA solution and allergen extract product and the concentration of PLGA. In one example, the matrix formulation is 60% 50:50 lactide/glycolide PLGA in in N-methyl pyrrolidone or DMSO and 0.5 mg/mL rapamycin. 1 ml of it is mixed with 0.1 mL Humira (40 mg/0.4 ml drug product) and the resulting final formulation can be injected to the patient to treat or prevent Humira ADA.

In some embodiments of the current inventions, the in-situ gelling matrix such as pectin, alginate, hyaluronic acid and gellan gum as described previously, rely on the gelling formation at the presence of di or trivalent cationic ion or polycationic molecule. In some embodiments, low water solubility di/trivalent/polycationic compound (e.g. low water solubility divalent cation salt such as calcium carbonate, calcium phosphate, dicalcium phosphate, calcium silicate, CaSO₄, ZnCO₃, BaCO₃, BaSO₄ or their combination), can be added to these type of in-situ gelling formulation right before injection, which can be injected when it is still in low viscosity state and forms gel slowly in vivo. In some embodiments, the final concentration of the low water solubility calcium or salt zinc salt or barium salt in the final drug loaded formulation to be injected is between 0.3-10%. A sustained release formulation that can release these cationic ions such as calcium ion or zinc ion or their combination slowly is also considered as a low water solubility cationic ion compound, e.g. nano or microparticle that can release its encapsulated calcium ion in 15 min-1 hr when it is in contact with water. Therefore the current invention also provide a kit to treat allergy, ADA or autoimmune diseases, which comprise two separate components in different containers to be mixed together right before injection. The two components can also be placed in one container if they are all in solid form (e.g. both being dried such as lyophilized). One component contains low water solubility di/trivalent/polycationic compound either in solid dosage form or liquid form that can enhance the gelling of the second component. The second component is an optional drug loaded in situ gelling formulation such as those described previously, or similar formulation with higher concentration of in situ gelling agent and optional drug, which will compensate the dilution factor upon mixing and provide same concentration of drug/gelling agent as those in the formulation previously described after it is mixed with the first component. In one example, the kit contains two components, one is 6% calcium carbonate or CaSO₄ or ZnCO₃ or calcium phosphate or dicalcium phosphate suspension in water, another is a previously optionally drug loaded formulation such as a saline solution containing 4% sodium alginate, optional immunosuppressant (such as 0.5-1 mg/mL rapamycin or methotrexate) and optional antigen. For example, the two components can also be mixed together with an antigen solution such as commercial allergen injection product or protein drug solution having ADA potential at 1:1:0.5 ratio and then the mixture is injected to treat said antigen related diseases. Additional viscosity enhancing polymer such as starch, cellulose, methyl cellulose, HPMC can also be incorporated into the first component at suitable concentration such as 0.1˜5% w/w. Alternatively, one of the components can contain the target antigen and optional immunosuppressant such as allergen or protein drug having ADA potential, therefor only component one and two will be mixed for injection. Adjuvant type agent such as 1-2 mg/mL poly IC or 0.5-1 mg/mL imiquimod can be used instead of the immunosuppressant if the formulation is for allergy treatment. In some embodiments, the immunosuppressant or disease related antigen or both can be in component 1 and component 2 contains gelling polymer only. The immunosuppressant can be in either component 1 or 2. The antigen can also be in either component 1 or 2, or in the commercially available product or mixing with antigen containing commercially available product is preferred.

In some embodiments, solution of water soluble (e.g. solubility >0.5% at room temperature) di/trivalent/polycationic compound (e.g. CaCl₂, calcium gluconate, Ca-EDTA, zinc chloride or gluconate, ferrous chloride FeCl₂, ferrous gluconate, FeCl₃, ferric gluconate, BaCl₂, barium gluconate, ornithine or its derivatives, lysine or its derivatives such as lysine ethyl ester, arginine or its derivatives such as arginine ethyl ester, chitosan, poly lysine, poly arginine, poly ornithine or their combination) can be injected into the same injection site right before or right after the site is injected with antigen/immunosuppressant loaded in-situ gelling formulation to improve the gelling effect. They can be also be co-injected using a dual syringe system. In some embodiments, the concentration of water soluble di/trivalent/polycationic compound in the solution is between 0.2-10%. In some embodiments, the concentration of the water soluble calcium salt in the solution is between 0.2-6%. When it is injected before the injection of the in-situ gelling agent containing formulation, it will prime the injection site with higher concentration of calcium ion than physiological calcium ion level to provide better in situ gelling effect. Therefore the current invention also provide a kit to treat allergy, ADA or autoimmune diseases, which comprise two separate components in different containers to be sequentially injected into one site or co-injected into one site using a dual syringe system. One component contains water soluble di/trivalent/polycationic compound that can enhance the gelling of the second component, either in solid dosage form or liquid form. The second component is a in situ gelling formulation such as those described previously, or similar formulation with higher concentration of in situ gelling agent and drug, which will compensate the dilution factor and provide same concentration of drug/gelling agent as those in the formulation previously described after it is mixed in vivo with the first component. In one example, the kit contains two components, the first one is 0.5-5% CaCl₂ or calcium gluconate or chitosan or lysine or arginine in water with pH value between 5-8, the second one is an in-situ gelling formulation, a saline solution containing 4% sodium alginate, optional immunosuppressant (such as 0.5-1 mg/mL rapamycin or methotrexate) and optional antigen. Additional viscosity enhancing polymer such as starch, cellulose, methyl cellulose, HPMC can also be incorporated into the first component at suitable concentration such as 0.1˜5% w/w. In one example, component 1 is a pH 7 solution containing 0.5% HPMC, 1% CaCl₂ or 2% calcium gluconate or 1.5% lysine or 1% chitosan, osmolarity adjusted with NaCl to be close to physiological value, is injected to a target site. Next the antigen and immunosuppressant loaded alginate containing solution component 2 is injected into the same site to treat the subject in need.

The component 1 can also be injected after the injection of component 2. Alternatively, the immunosuppressant or disease related antigen or both can be in component 1 and component 2 contains gelling polymer only. The immunosuppressant can be in either component 1 or 2. The antigen can also be in either component 1 or 2, or in the commercially available product or mixing with antigen containing commercially available product is preferred. Adjuvant type agent such as 1-2 mg/mL poly IC or 0.5-1 mg/mL imiquimod can be used instead of the immunosuppressant if the formulation is for allergy treatment.

Component 1 and 2 can also be placed in one syringe for injection, separated by a biologically and pharmaceutically acceptable liquid solution as a buffer layer to prevent them being mixed together inside the syringe. In some embodiment, the buffer layer liquid can be a liquid having high viscosity (e.g. >500 cps , or >2000 in other embodiments) to reduce permeation and contamination between components 1 and 2. For example, it can be glycerin, or a pH 7 solution containing viscosity enhancing polymer such as starch, cellulose, methyl cellulose, HPMC, HA at 0.3˜3% w/w, osmolarity adjusted to 250˜350 mOsm/kg with NaCl. In one example, a syringe containing 0.5 ml of component 1 and 0.5 ml component 2 with 0.3 ml buffer liquid in the middle is used for injection. Therefore, the kit to treat allergy, ADA or autoimmune diseases can further comprise a 3rd component, which is a buffer layer liquid as described above.

In some embodiments, solution of water soluble (e.g. solubility >0.5% at room temperature) di/trivalent/polycationic compound can also be mix with the alginate containing formulation first and then being injected. Viscosity enhancing polymer such as starch, cellulose, methyl cellulose, HPMC can also be incorporated in either component at suitable concentration such as 0.1˜5% w/w to prevent the gelling before injection. Another approach to slow and control gelation is to utilize a buffer containing phosphate (e.g., sodium hexametaphosphate), as phosphate groups in the buffer compete with carboxylate groups of alginate in the reaction with calcium ions, and retard gelation as sequestrant. Typical sequestrants that can be employed include a variety of inorganic phosphates such as sodium hexametaphosphate, tetrasodium pyro-phosphate, disodium orthophosphate, and sodium tripolyphosphate. Sodium citrate can also be used.

In some embodiments, the low water solubility divalent cation salt (e.g. calcium carbonate, calcium phosphate, dicalcium phosphate, calcium silicate, CaSO₄, ZnCO₃, BaCO₃, BaSO₄ or their combination) or Ca-EDTA containing formulation can further mix with a agent that can slowly release these cation from the low solubility salt or from Ca-EDTA complex right before administrate the formulation to a subject in need, which will cause the gelling slowly in vivo.

Example of these agent can be selected from D-glucono-delta-lactone (GDL), L-glucono-delta-lactone, D-erythronolactone, L-erythronolactone, D-glucuronolactone, L-glucuronolactone, D-galactono-gamma-lactone, L-galactono-gamma-lactone, D-xylono-gamma-lactone, L-xylono-gamma-lactone, D-gulono-gamma-lactone, L-gulono-gamma-lactone 3, D-glucono-gamma-lactone and L-glucono-gamma-lactone. Those lactone can hydrolyze slowly in water to release acid which will release the free divalent cation in to water to cause gelling. Higher pH increase hydrolysis speed and lower pH reduce hydrolysis speed which will in turn affect the gelling time. The pH of the formulation can be adjusted accordingly (e.g. pH5-8) to achieve the desired gelling time. The ratio of these agent vs divalent cation salt can be between 1:5 to 5:1 molar ratio. For example, when GDL and CaCO₃ or mixture of CaCO₃ with CaSO₄ are used in the formulation, their molar ratio (GDL: calcium salt) can be 1:2 or 1:1 or 2:1. In one example, 5 mL drug loaded ormulation containing 2% sodium alginate is mixed with 0.1 g CaCO₃ powder and then mixed with 0.05 g GDL powder. After stirring, the final formulation is injected to form gel in vivo.

In one example, the first formulation is an in-situ gelling formulation, a saline solution containing 4% sodium alginate, optional immunosuppressant (such as 0.5-1 mg/mL rapamycin or methotrexate) and suitable amount of disease related antigen (e.g. allergen or autoantigen causing autoimmune disease or drug that producing ADA); additional viscosity enhancing polymer such as starch, cellulose, methyl cellulose, HPMC can also be incorporated into the first component at suitable concentration such as 0.1˜5% w/w. 5 mL the first formulation containing 2% sodium alginate is mixed with 0.1 g CaCO₃ powder and 0.05 g GDL powder. After stirring, the final formulation is being injected to the subject in need as subcutaneous injection or intramuscular injection or intralymphatic injection to form gel in vivo.

In another example, the first formulation is 2.5 mL of saline solution containing 4% sodium alginate and optional 2 mg/mL poly IC or optional 0.5 mg/mL rapamycin, the second formulation is a dry powder mixture of 0.05 g CaCO₃ (or ZnCO₃), 0.05 g CaSO₄ and 0.05 g GDL (or L-gulono-gamma-lactone). Additional viscosity enhancing agent can be incorporated in the first or second or both formulation, which will reduce the gelling speed. Suitable strength of 0.5 mL Alutard SQ dust mite extract (e.g. 10 Allergy Unit/mL dilution) is mixed with the first and second formulation to generate a mixture and the resulting final formulation can be injected to the patient to treat dust mite allergy. The first formulation can also be in dry form such as lyophilized form together with suitable bulking agent/lyoprotectant and the dried formulation 1 is placed together with formulation 2 powder in the same vial. The user only needs to add Alutard SQ dust mite extract solution to the vial to form the final formulation for injection.

In another example, the first formulation is 2.5 mL of saline solution containing 4% sodium alginate, 5 mg/mL adalimumab and 1 mg/mL rapamycin, the second formulation is a dry powder mixture of 0.05 g CaCO3(or ZnCO3), 0.05 g CaSO4 and 0.05 g GDL (or L-gulono-gamma-lactone). Additional viscosity enhancing agent can be incorporated in the first or second or both formulation, which will reduce the gelling speed. The first and second formulation is mixed to generate a mixture and the resulting final formulation can be injected to the patient to prevent or treat ADA against adalimumab. The first formulation can also be in dry form such as lyophilized form together with suitable bulking agent/lyoprotectant and the dried formulation 1 is placed together with formulation 2 powder in the same vial. The user only needs to add diluent (e.g. PBS or water) to the vial to form the final formulation for injection.

The antigen/drug loaded sustained release formulation in the current inventions can be either in-situ gelling formulation or nano/microparticles based formulation or their combinations. The drug (e.g. immunosuppressant or adjuvant type agent) and antigen can be encapsulated in nanoparticle or microparticle as a sustained release form to be injected. In some embodiments, the drug/antigen encapsulated nanoparticle or microparticle is polysaccharide such as alginate based particle which use polysaccharide such as alginate to form the matrix of particle. For example, it can be chitosan-calcium alginate gel nano/microsphere such as those described in patent number CN1628861A, or chitosan-alginate nano/microsphere, or calcium alginate nano/microsphere such as those described in patent application number CN107057085A, or sodium alginate-calcium carbonate hybrid microparticles such as those described in patent application number CN102286155A, or calcium phosphate/calcium alginate hybrid microspheres such as those described in patent application number CN101081911A. These antigen/drug loaded particles can be readily prepared by using an alginate solution containing antigen/drug by adopting or modifying the protocols described in prior publications.

In one example, dissolve 100 mg of sodium alginate in 10 ml of distilled water, heat in a water bath at 40° C., add 300 mg solution of sodium phosphate and stir in a water bath for 30 minutes, add 5 mg rapamycin, 50 mg adalimumab, then slowly add 5ml 1% calcium chloride solution and stir in an hour, then centrifuge, to obtain antigen/drug loaded alginic acid-calcium phosphate microparticles to inhibit ADA against adalimumab. In another example, drug loaded alginate-Ca particle is prepared by adding drug containing 2.0% (w/v) sodium alginate solution (e.g. 5 mg/mL poly IC or 1 mg/mL imiquimod or 1 mg/mL rapamycin) and 10 mg/mL gluten or egg white protein in alginate solution using electrostatic drop method and vigorous stirring to 1% CaCl₂ solution as the gel bath to obtain calcium alginate gel microspheres, then the antigen/drug loaded calcium alginate gel microspheres is coated with 0.7% (w/v) chitosan solution by mixing them at 1:10 v/v ratio. Then centrifuge to obtain antigen/drug loaded alginic-calcium-chitosan microparticles.

Skin patches containing allergen such as those developed by DBV Technologies are used to treat allergy by inducing tolerance for the antigen (allergen). The topically patch can be readily adopted for the current application. For example, the topical applied formulation such as patch described in US patents U.S. Pat. No. 6,676,961, 8,932,596B2 and 8,202,533B2 can be adopted for the current application by adding additional immune suppressive drug in the patch (e.g. 0.05 mg˜5 mg of rapamycin or fujimycin or 0.1 mg-10 mg methotrexate or their directives or prodrug) as well as those commercial available patch (e.g. VIASKINR MILK and VIASKINR PEANUT). The administration method can be essentially the same as the prior arts except the patch contains immunosuppressants. Similar patches are also described in U.S. application Ser. No. 15/723,173, Ser. No. 16/380,951, Ser. No. 16/029,594, Ser. No. 17/344,932, Ser. No. 16/566,716, Ser. No. 16/819,168 and Ser. No. 17/385,908. Additional transdermal enhancer (e.g. DMSO, Azone, fatty acid, hyaluronic acid and etc., which can be found in the publication readily as well as their suitable amount) can be added to the patch or applied to the skin before applying the patch. Example of transdermal enhancing agent can be added include DMSO (e.g. 10˜300 mg/patch), azone (e.g. 1%˜10% of total drug weight), surfactant, fatty acid (e.g. 1%˜10% oleic acid). The skin stratum corneum can also be removed with exfoliation or other means to enhance the transdermal delivery. In one example, the patch contains 500 μg-10 mg gluten (e.g. G5004 gluten from wheat, Sigma) and 0.1 mg˜2 mg of rapamycin or 0.1 mg-5 mg methotrexate. For example, antigen such as gluten and immunosuppressant such as rapamycin and/or methotrexate can be in powder form, which can be simply mixed together physically, they can also be co-dissolved and then dried and then placed in the patch. In another example, the patch contains 5 mg gluten (e.g. G5004 gluten from wheat, Sigma) and 0.5 mg of rapamycin or 0.5 mg methotrexate and optionally additional 30 mg azone. In another example, the patch contains 5 mg gluten (e.g. G5004 gluten from wheat, Sigma) and 100 mg of sialic acid or sialic acid-cholesterol conjugate or 2 mg methotrexate. This can be used to induce gluten tolerance and treat gluten intolerance. The gluten can be replaced with deamidated gliadin instead. In embodiments, the patch can be applied daily for 1-5 weeks. In another example, the antigen is peanut antigen ara h2 200 μg and 0.2 mg of rapamycin is in the patch to treat peanut allergy. In one example, peanut antigen ara h2 200 μg, 0.2 mg of rapamycin and 50 mg sucrose is dissolved in water and then lyophilized and then placed in the patch. In one example, peanut antigen ara h2 200 μg, 0.5 mg of rapamycin, 50 mg SDS and 50 mg sucrose is dissolved in water and then lyophilized and then placed in the patch. In one example, peanut antigen ara h2 200 μg, 0.2 mg of rapamycin, 100 mg DMSO and 50 mg sucrose is dissolved in water and then lyophilized and then placed in the patch. In another example, the antigen is the double strand DNA (1 mg˜10 mg) in the previous figures to treat lupus and the drug is 1 mg of rapamycin or fujimycin or temsirolimus. In another example, the nasal spray contains 1 mg gluten (e.g. G5004 from Sigma, gluten from wheat) and 0.5 mg of rapamycin or 1 mg methotrexate in a suitable form for each spray. In another example, the sublingual lozenge contains 50 mg gluten (e.g. G5004 from Sigma, gluten from wheat) and 0.1-0.5 mg of rapamycin or 0.1-0.5 mg methotrexate. In another example, the gel contains 50 mg gluten (e.g. G5004 gluten from wheat, Sigma) and 0.2 mg of rapamycin or 1 mg methotrexate in each 1 ml of gel. The immunosuppressant drug or both the immunosuppressant drug and the antigen can be either in the form of powder or gel or semi liquid or in the form of liposome (e.g. 100 nm˜5 μm diameter) or in a nano/micro particle (e.g. 100 nm˜1 μm) or being conjugated to a dendrimer or linear polymer (e.g. couple to poly acrylic acid or poly Sialic acid via ester bond to form a polymer based prodrug with MW=5KD˜500KD). Other pharmaceutically acceptable amount of antigen and immunosuppressant can also be used in the patch, as long as it can produce satisfactory biological and therapeutical (e.g. immune tolerance) effect, which can be determined experimentally by screening and testing with well-known protocol and methods. Other allergen such as pollen extract, dust mite extract, animal hair extract and food allergen such as nut/peanut/milk/egg extract can also be used instead in the above formulations to treat related allergy. Example of the concentration of these allergens can be between 0.1 mg to 1 mg/patch as long as they are tolerable by the subject in need. During the treatment, the concentration of the allergen in the formulation can increase when the patient's tolerance increases, which is the standard practice of the allergy immune therapy.

Pharmaceutically acceptable amount of antigen causing allergy and immune activity enhancing agent can be used in the patch instead of using antigen and immunosuppressant in the patch. In some embodiments, the method is to use a patch containing both allergen or its fragment and immune enhancing agent described previously (such as imiquimod or poly IC) as a mixture. It can also contain anti-allergy drug such as antihistamines, corticosteroids, mast cell stabilizers, and leukotriene inhibitor. A mixture of allergy causing antigen and immune activity enhancing agent can be a physical mixture. A physical mixture means that the mixture of antigen and immune activity enhancing agent are simply mechanically mixed (e.g. by stirring or blending) together in their original form (e.g. liquid or solid form such as powder or particles) without any additional process (e.g. by mixing them in their original form together), or further size reducing process is applied after the mechanical mixing (e.g. crashing, grinding, mulling or homogenizing), or dispersed or dissolved separately in same or different type of liquid and then mix, or co-dispersed in liquid, or co-dissolved in solvent (e.g. water), and optional drying process (e.g. spray drying or lyophilization) can be applied with optional further size reducing process.

For example, the topical applied formulation such as patch described in US patents U.S. Pat. No. 6,676,961, 8,932,596B2 and 8,202,533B2 can be adopted for the current application by adding additional immune enhancing drug (e.g. 0.1 mg˜20 mg of imiquimod or poly IC or their directives or prodrug) into the patch or those commercial available patch (e.g. VIASKINR MILK and VIASKINR PEANUT). The administration method can be essentially the same as the prior arts except the patch contains immune activity enhancing agents. Additional transdermal enhancer such as those described previously can be added to the patch or applied to the skin before applying the patch. Example of transdermal enhancing agent can be added include DMSO (e.g. 10˜300 g/patch), azone (e.g. 1%˜10% of total drug weight), surfactant, fatty acid (e.g. 1%˜10% oleic acid). In one example, the patch contains 500 μg˜10 mg gluten (e.g. G5004 gluten from wheat, Sigma) and 0.05 mg˜5 mg of imiquimod or 0.05 mg˜5 mg R848. For example, allergy causing antigen such as gluten and immune activity enhancing agent such as imiquimod and/or poly IC can be in powder form, which can be simply mixed together physically, they can also be co-dissolved and then dried and then placed in the patch. For example, 10 mg gluten powder and 0.25 mg of imiquimod powder are blended and then homogenized with a grinder, and then applied to the surface of the skin contact side of a 5×5 cm2 dermal patch. In another example, 10 mg gluten and 10 mg of poly IC are mixed in 10 mL water containing 30 mg sucrose vigorously for 10 min and then lyophilized, and then the dry mixture is applied to the surface of the skin contact side of a 5×5 cm2 dermal patch. In another example, 10 mg gluten, 5 mg of STING agonist MK-1454 and 5 mg of CpG ODN are dissolved in 5 mL 25% EtOH water solution and then vacuum dried, and then the dry mixture is placed to the surface of the skin contact side of a 3×3 cm2 dermal patch. In another example, 10 mg gluten and 0.5 mg of imiquimod are dissolved in 5 mL 1% SDS water solution and then vacuum dried, and then the dry mixture is placed to the surface of the skin contact side of a 3×2 cm2 dermal patch. In another example, the 10×10 cm2 patch contains 5 mg gluten (e.g. G5004 gluten from wheat, Sigma) and 0.5 mg of imiquimod or 0.1 mg 3M-052 and optionally additional 30 mg azone. In another example, the patch contains 5 mg gluten (e.g. G5004 gluten from wheat, Sigma) and 100 mg of sialic acid or sialic acid-cholesterol conjugate and 1 mg poly IC. This can be used to induce gluten tolerance and treat gluten intolerance. The gluten can be replaced with gliadin instead. In embodiments, the gluten or gliadin containing patch can be applied daily to forearm 8 hours a day for 1-5 weeks. The gluten in the above examples can be replaced with egg white protein such as 5-10 mg of ovomucoid (Gal d 1) or 5-10 mg ovalbumin (Gal d 2) or their combination with optional 5-10 mg ovotransferrin (Gal d 3) and 5-10 mg lysozyme (Gal d 4) to treat egg white allergy. In another example, the antigen is peanut antigen ara h2 200 μg and 0.5 mg of imiquimod is in the patch to treat peanut allergy. In one example, peanut antigen ara h2 200 μg, 0.2 mg of imiquimod and 50 mg sucrose is dissolved in water and then lyophilized and then placed in the patch. In one example, peanut antigen ara h2 200 μg, 0.2 mg of imiquimod, 50 mg SDS and 50 mg sucrose is dissolved in water and then lyophilized and then placed in a 5×5 cm2 patch. In one example, peanut antigen ara h2 200 μg, 0.2 mg of imiquimod, 100 mg DMSO and 50 mg sucrose is dissolved in water and then lyophilized and then placed in the patch. In another example, the nasal spray or nasal drop contains 1 mg gluten (e.g. G5004 from Sigma, gluten from wheat) and 0.05 mg of imiquimod or 0.5 mg poly IC in a suitable form for each spray or every 3 drops, viscosity enhancing agent can be added, such as hyaluronic acid or carbomer. In another example, the sublingual lozenge contains 50 mg gluten (e.g. G5004 from Sigma, gluten from wheat) and 0.1 mg of imiquimod or 2 mg poly IC. In another example, a gel contains 50 mg gluten (e.g. G5004 gluten from wheat, Sigma) and 0.2 mg of imiquimod or 2 mg poly IC in each 1 ml of gel. The immune activity enhancing agent drug or both the immune activity enhancing agent drug and the allergy causing antigen can be either in the form of powder or gel or semi liquid or in the form of liposome (e.g. 100 nm˜5 μm diameter) or in a nano/micro particle (e.g. 100 nm˜1 μm) or being conjugated to a dendrimer or linear polymer (e.g. couple to poly acrylic acid or poly Sialic acid via ester bond to form a polymer based prodrug with MW=5 KD˜500 KD).

In some embodiments, the topical formulations contain 0.1˜10 mg allergen, 0.01˜5 mg TLR7/8 ligands (e.g. imiquimod or gardiquimod or resiquimod), 0.1˜5 mg TLR3/RLR ligands (e.g. dsRNA such as poly IC or polyICLC), 0.1˜5 mg TLR9 ligands (e.g. CpG ODNs such as ODN 1826 or ODN 2216) in each patch or each mL of gel or lotion or liquid. Transdermal enhancing agent can be added to it as well such as DMSO, azone (e.g. 1%˜10%), surfactant, fatty acid (e.g. 1%˜10% oleic acid). In one example, the formulations contain 5 mg/mL gluten, 0.5 mg/mL imiquimod, 1 mg/mL poly IC, 1 mg/mL class A CpG ODN 2216, 20 mg/mL SDS in 1× PBS and 5% sucrose and then being lyophilized. The lyophilized powder can be used to prepare a skin patch and attached to the skin at 10˜500 mg powder/patch. In another example, 10˜100 mg egg white powder, 0.1-0.5 mg of imiquimod, 1-5 mg of poly IC and 5-50 mg of azone is mixed together and added to a Viaskin® like dermal patch. It can be applied to the skin twice every week for 2 weeks, each time for 2 day and then applied for 2 days as a booster after 1 month and 3 month to generate egg tolerance. In another example, 10 mg peanut protein, 0.5 mg of imiquimod, 2 mg of poly IC and 100 mg of DMSO is mixed together and added within a Viaskin® like device. It can be applied to the skin twice every week for 2 weeks, each time for a and then applied for 2 day after 1 month and 3 month to generate peanut tolerance. In some embodiments, the topical formulations contain 0.1˜100mg antigen, 0.05˜5 mg TLR agonist in each mL of gel or lotion or liquid; transdermal enhancing agent can be added to it as well such as DMSO, azone (e.g. 1%˜10%), surfactant, fatty acid (e.g. 1%˜10% oleic acid).

The formulation can also be an oral formulation such as a tablet or capsule containing the mixture of allergy causing antigen and immune enhancing agent. It can be the same as those used by Aimmune's oral formulation (e.g. its AR101 for peanut allergy) except additional said immune enhancing agent is added. Viscosity enhancing agent can also be incorporated similar to those described above. It can also contain therapeutically effective amount (e.g. the dose currently used in clinic) of anti-allergy drug such as antihistamines, corticosteroids, mast cell stabilizers, and leukotriene inhibitor as described above. The addition of these anti-allergy drugs can prevent the allergy reaction induced by giving the allergen to the patient. In one embodiment, a formulation is an enteric capsule containing 1 mg-100 mg peanut protein, 1-10 mg imiquimod, 10 mg carbomer 940 and 10 mg cetirizine. At later stage treatments, the peanut protein amount is increased as those used in AR101. In some embodiments, the immune enhancing agent can be replaced with silica acid or poly sialic acid or sialic acid polymer or siglec ligand or their derivatives (e.g. 50˜500 mg/capsule).

In some embodiments, the formulation is applied to oral mucosa. It can be suppository, lozenge, tablet, film used for sublingual delivery and oral mucosa delivery. Current formulation used for sublingual delivery and oral mucosa delivery of allergen (e.g. pollen extract, dust mite extract) can be used with additional immune activity enhancing agent added to the formulation. Viscosity enhancing agent and/or mucosa adhesive agent can also be incorporated as described above. It can also contain therapeutically effective amount (e.g. the dose currently used in clinic) of anti-allergy drug such as antihistamines, corticosteroids, mast cell stabilizers, and leukotriene inhibitor as described above. It can also be a non-biodegradable container (e.g. a tablet made with plastic or metal) with small holes that allow the enclosed allergen/drugs to be released to the oral mucosa once it is placed in the mouth instead of the orally dissolvable tablet/lozenge from. A means that can prevent the formulation from being swallowed can be incorporated to the delivery system, such as a string, a band, a stick or tooth retainer. This will allow the removal of the formulation from mouth easily when severe allergy reaction is shown. For example, it can be in a format of a lollipop with the lozenge containing both allergen and other drugs. In one embodiment, a formulation is a mucosa adhesive tablet containing 1 mg-100 mg peanut protein, 0.1-1 mg imiquimod or poly IC, and 3 mg cetirizine with a wood handle attached in a lollipop format.

For transdermal/transmucosal delivery or implant type formulation or oral formulation or sustained release formulation, the initial amount of allergy causing antigen and the amount of immune activity enhancing agent can be between 0.1-100 mg. When injection or non-sustained release oral formulation is used, the initial amount of allergen can be between 50 μg-5 mg and the amount of immune activity enhancing agent can be between 0.05-5 mg.

The formulation/composition can contain increased dose of allergen in later stage similar to the dosing protocol used by current treatment protocol using allergen (oral or topical or injection). That is, the treatment involves a series of formulations, the first formulation contains lowest amount of allergen and it gradually increases over time in the later formulation while the amount of other drug (e.g. immune enhancing agent) can be unchanged. The allergen amount in the first formulation can be the highest amount of allergen that can be tolerated by patient without causing severe allergenic reaction. In one example, the patient began with a first single dose of oral mucosal tablet formulation containing 0.1 mg of powdered egg white and 0.2-2 mg of imiquimod, after the initial dose, subject received approximately doubling doses of egg white but same amount of imiquimod every 30 minutes until the highest tolerated single dose is determined (as shown in table 1). Based on the highest tolerated single dose, subject begins daily dosing with between the formulation containing powdered egg white and 0.2-2 mg imiquimod, 1 dose daily for 2 weeks. As long as subject is tolerating current doses, the egg white powder in the formulation containing 5 mg imiquimod are increased by 25 mg every 2 weeks until reaching 150 mg and then increased by 50 mg every 2 weeks until reaching 300 mg. Once subjects reached the daily dose of 300 mg, they are instructed to take this daily dose that does not contain imiquimod for 2 years.

The current invention also discloses an autologous immune cell therapy method to treat autoimmune disease, allergy, inhibit anti-drug antibody production or induce antigen specific immune tolerance in a subject. It comprises the following steps: autologous immune cell collection and separation from a subject in need, stimulating with disease related antigen and immunosuppressant to expand antigen specific regulatory immune cell and/or inhibitory immune cells in vitro including tolerogenic DC cell to reach a desired number of target cells, and then infuse back the expanded autologous immune cell to the subject for desired therapeutical effect. The source of autologous immune cell collection and separation from a subject can be bone marrow or lymph node extract or blood or blood fraction from the said subject or their combinations. In some embodiments, one can separate the lymphocyte from the blood of the subject in need with blood cell separator and/or leukapheresis. For example, 200 ml blood is draw from the patient and the lymphocyte is collected by using a blood cell separator on this 200 ml blood. The procedure of lymphocyte collection from blood is well known to the skilled in the art. It can be performed using commercial blood cell separator. The resulting lymphocyte contains B cell and T cell and possibly other white blood cells. Optionally the B cell can be further removed, e.g. with a cell sorter such as FACS or magnetic particles coated with B cell surface marker specific antibody, there are many commercial kits and instruments available for this purpose and the procedure is well known to the skilled in the art. However, in other embodiments the B cells are desired to stay in order to convert them to Breg cells. In the current invention inhibitory immune cells that can inhibit immune function is considered as regulatory immune cell, therefore regulatory immune cell includes both antigen specific regulatory immune cell and none- antigen specific inhibitory immune cells.

In some embodiments, the collected immune cell contains DC cells, T cell, and B cell. In some embodiments, the collected immune cell contains DC cells and T cells but no B cell. In some embodiments, the collected immune cell contains DC cells and no T cell/B cell.

The culture medium contains diseases related antigen and immunosuppressant (e.g. rapamycin, IL-10, IL-2/anti-IL-2 mAb, PD-L1, which can be found in U.S. application Ser. No. 16/566,716 by the current inventor). Preferably the antigen is the antigen in their natural form or their peptide fragment but not in MHC bound complex form. For example, it can be dust mite extract or pollen extract or food allergen (e.g. peanut protein, gluten) to treat related allergy. It can be protein drug to treat related ADA. It can be double strand DNA to treat lupus. It can be antigen protein to treat related autoimmune disease (e.g. PPI, IGRP, GAD, islet cell autoantigen-2, insulin, insulin receptor to treat diabetes; collagen to treat rheumatoid arthritis). The cell culture protocol can be readily adopted from prior U.S. application Ser. No. 16/566,716 and well-known publications. For example, the procedure of DC cell in vitro culture to treat cancer is well established, same protocol can be adopted by using diseases related autoantigen instead of tumor antigen, and adding tolerogenic immune cell inducing immunosuppressant into the culture medium. TLR agonist can also be added to the culture medium to stimulate more than 24 hrs to induce DC cell exhaustion.

For example, the concentration of antigen (e.g. peanut protein or collagen type II) can be between 0.1 m/ml to 10 mg/ml in the culture media. In one example, the collected DC cells from the subject is cultured in complete medium, which consisted of 10% heat-inactivated fetal bovine serum (Biosource International), nonessential amino acids, 0.5 mM sodium pyruvate, 5 mM Hepes, 1 mM glutaMax I (all from Invitrogen), gluten as antigen at 50 μg/ml in DMEM base. The culture is monitored daily and maintained at 0.7˜1×10⁶/ml by diluting with complete medium for 8-12 days or until desired amount of target cells are obtained. 200-2000IU/mL IL-2/anti-IL-2 mAb (e.g. those described in DOI: 10.4049/jimmuno1.1402540) is included into the medium. 2-20 ng/mL rapamycin is also included in the culture medium.

Alternative, allogenic cell can be used instead. The DC cells from a healthy donor is cultured the same way and then transferred to a subject in need.

Compounds and compositions (e.g. the composition, conjugate, polymer and nano/micro particle disclosed in the current invention) described herein can be administered as a pharmaceutical or medicament formulated with a pharmaceutically acceptable carrier. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from portion of the body, to another portion of the body, or to deliver an agent to the desired tissue or a tissue adjacent to the desired tissue. Pharmaceutically acceptable carriers are known to one having ordinary skill in the art may be used, including water or saline. As is known in the art, the components as well as their relative amounts are determined by the intended use and method of delivery. Diluent or carriers employed in the compositions can be selected so that they do not diminish the desired effects of the composition. Examples of suitable compositions include aqueous solutions, for example, a saline solution, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents. In certain embodiments, polymeric material is employed as a pharmaceutically acceptable carrier. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, compositions provided herein may be formulated as films, gels, foams, or and other dosage forms. Suitable ionic strength modifying agents include, for example, glycerin, propylene glycol, mannitol, glucose, dextrose, sorbitol, sodium chloride, potassium chloride, and other electrolytes. Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES. In certain embodiments, the pH adjusting agent (e.g. HCl, HePO4, NaOH) and/or pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described. The exact formulation, route of administration and dosage can be chosen by the individual physician in view of the patient's condition.

In the current application, the “I” mark means “and” and/or “or” and/or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from textbooks such as the chemistry textbooks, scientific journal papers and other well-known reference sources.

1. A composition to induce immune tolerance to an antigen comprising an antigen causing immune intolerance and an immunosuppressant in a self-gelling formulation.

2. The composition according to claim 1, wherein the immunosuppressant is rapamycin.

3. The composition according to claim 1, wherein the antigen is an allergen.

4. The composition according to claim 1, wherein the self-gelling formulation comprises alginate and calcium ion.

5. The composition according to claim 1, wherein the self-gelling formulation comprises PLGA and N-methyl pyrrolidone.

6. A composition to induce immune tolerance to an antigen comprising an antigen causing immune intolerance and an immunosuppressant in a sublingual formulation.

7. The composition according to claim 6, wherein the immunosuppressant is rapamycin.

8. The composition according to claim 6, wherein the antigen is an allergen.

9. The composition according to claim 6, wherein the sublingual formulation is sublingual drop.

10. The composition according to claim 6, wherein the sublingual formulation is sublingual tablet.
